(19) United States
(12) Patent Application Publication
(10) Pub. No.: US 2024/0409607 A1
(43) Pub. Date: Dec. 12, 2024

(54) STABILIZED MHC MOLECULES

(71) Applicant: Immatics Biotechnologies GmbH, Tuebingen (DE)

(72) Inventors: Thomas TREIBER, Tuebingen (DE); Stefanie SPALT, Tuebingen (DE); Dominik MAURER, Tuebingen (DE)

(21) Appl. No.: 18/701,949

(22) PCT Filed: Oct. 18, 2022
(86) PCT No.: PCT/EP2022/078967

§ 371 Date: Apr. 17, 2024

Related U.S. Application Data
(60) Provisional application No. 63/256,965, filed on Oct. 18, 2021.

(30) Foreign Application Priority Data
Oct. 18, 2021 (EP) 21203254.4

(51) Int. Cl. C07K 14/74; A61K 9/08; A61K 47/12

(52) U.S. Cl. CPC C07K 14/70539; A61K 9/08; A61K 47/12

(57) ABSTRACT

The present invention relates to a composition comprising a major histocompatibility complex (MHC) protein, and an R₁—COOH or salt thereof. The invention further relates to the use of an R₁—COOH or salt thereof, for stabilizing an MHC protein. The invention also relates to a method for stabilizing an MHC protein. The invention also relates to a kit comprising an MHC protein, and an R₁—COOH or salt thereof.

The present invention relates to a composition comprising a major histocompatibility complex (MHC) protein, and an R₁—COOH or salt thereof. The invention further relates to the use of an R₁—COOH or salt thereof, for stabilizing an MHC protein. The invention also relates to a method for stabilizing an MHC protein. The invention also relates to a kit comprising an MHC protein, and an R₁—COOH or salt thereof.

### BACKGROUND OF THE INVENTION

Major Histocompatibility Complex (MHC) proteins class I or class II present peptide ligands derived from pathogens or endogenous proteins on the cell surface of antigen presenting cells for recognition by cytotoxic T cells (Rodenko B. et al.: “Generation of peptide-MHC class I complexes through UV-mediated ligand exchange”; Nature Protocols; vol. 1, no.: 3, 1120-1132; 2006). MHC class I complexes presenting tumor-associated peptides such as neoantigens represent key targets of cancer immunotherapy approaches currently in development, rendering them important for efficacy and safety screenings. Without peptide ligand, MHC class I complexes are unstable and decay quickly, making the production of soluble monomers for analytical purposes labor intensive (Moritz A. et al.: “High-throughput peptide-MHC complex generation and kinetic screenings of TCRs with peptide-receptive HLA-A*02:01 molecules”; Science Immunology, 4, 2019). Stabilization of MHC molecules can be achieved by introducing an artificial disulfide bridge into the MHC (e.g. Moritz A. et al., 2019). Dipeptides consisting of glycine and methionine may further stabilize the MHC molecules, e.g. by refolding the MHC molecule in the presence of a dipeptide (Anjanappa R. et al.; “Structures of peptide-free and partially loaded MHC class I molecules reveal mechanism of peptide selection”, Nature Communications; 11:13141; 2020). Nevertheless, there remains a need to stabilize empty MHC molecules or compositions thereof for biology applications or high-throughput screening assays, for example, to only name a few.

The objective technical problem is solved by the herein provided invention. As set out below, the invention inter alia relates to a composition comprising (i) a major histocompatibility complex (MHC) protein; (ii) an R₁—COOH or salt thereof; wherein R₁ is substituted or unsubstituted hydrocarbyl. It was surprisingly demonstrated in the examples that the R₁—COOH or salt thereof stabilized the MHC protein. It was shown that various lengths of the R₁—COOH (different number of carbon atoms) stabilized the MHC protein. In addition, linear, branched, saturated and unsaturated R₁—COOH stabilized the HLA molecule as shown in the Examples. The Examples further document that stabilization is observed for different MHC proteins. Furthermore, it was unexpectedly shown that R₁—COOH even further stabilized MHC proteins bound to the dipeptide “GM” consisting of glycine and methionine.

The R₁—COOH or salt thereof is usually readily available in laboratories and is cheaper compared to the above-mentioned dipeptides. Accordingly, the present invention provides advantageous means to stabilize MHC proteins, and its uses. The present invention provides inter alia further advantages: (i) MHC proteins or compositions thereof that can be readily loaded with a peptide of interest, preferably the MHC proteins are peptide free or loaded with very short easily replaceable peptides; (ii) MHC proteins or compositions thereof are amenable to long term storage either at 4° C. or frozen; (iii) MHC proteins or compositions thereof are amenable to high-throughput screening of T-cells, T cell receptors (TCRs) or TCR-like ligands, i.e. ligands that recognize peptide-MHC complexes.

### SUMMARY OF THE INVENTION

In a first aspect the invention relates to a composition comprising
(i) a major histocompatibility complex (MHC) protein; and
(ii) an R₁—COOH or salt thereof, wherein R₁ is substituted or unsubstituted hydrocarbyl.

In a second aspect the invention further relates to the use of an R₁—COOH or salt thereof, for stabilizing a MHC protein, wherein R₁ is substituted or unsubstituted hydrocarbyl.

A third aspect of the invention relates to a method for stabilizing an MHC protein, comprising the following steps:
(i) providing a solution comprising an MHC protein;
(ii) contacting said solution with R₁—COOH or salt thereof,
wherein R₁ is substituted or unsubstituted hydrocarbyl.

A fourth aspect of the invention relates to a kit comprising:
(i) an MHC protein; and
(ii) an R₁—COOH or salt thereof, wherein R₁ is substituted or unsubstituted hydrocarbyl.

### LIST OF FIGURES

In the following, the content of the figures comprised in this specification is described. In this context please also refer to the detailed description of the invention above and/or below.

FIG. 1: Melting points of HLA-A*02:01_ds 22-71 measured by thermal shift assay (TSA). Data points for 33 additives specified in Table 3 and a control sample are plotted. Points for sodium butyrate and GM dipeptide additives are labelled.

FIG. 2: Melting points of HLA-A*02:01_ds 22-71 measured by TSA with fatty acid additives as indicated. Values are average of duplicate measurements.

FIG. 3: Melting points of HLA-A*02:01_ds 22-71 measured by TSA with addition of 50 mM of linear and branched chain fatty acids. Used compounds are: 5C: valeric acid, isovaleric acid; 6C: hexanoic acid, 4-methyl-valeric acid. Values are average of duplicate measurements.

FIG. 4: Melting points of HLA-A*02:01_ds 22-71 measured in TSA with 50 mM of fatty acid additives as indicated. Values are average of duplicate measurements.

FIG. 5: Melting points of HLA-A*02:01_ds 22-71 or HLA-A*02:01_ds 84-139 measured in TSA with fatty acid additives as indicated. Values are average of duplicate measurements.

FIG. 6: Panel A shows the melting curves of HLA*A02:01 ds 22-71. Shown is the change of fluorescence in a temperature gradient measured by TLA. Fatty acid and Gly-Met (GM) dipeptide additives were included in the samples as indicated in the legend in the Figure itself. Data shown is the average of triplicate measurements. Panel B shows the melting curves of HLA*A02:01 ds 39-184 measured as in A. Panel C is a table of melting points of HLA-A*02:01_ds 22-71 or HLA-A*02:01_ds 84-139 derived from the data shown in panels A and B.

FIG. 7: Panel A shows the melting points of HLA-A*02:01_ds 22-71 measured in TSA with fatty acid and dipeptide additives as indicated. Panel B shows the melting points of HLA-A*02:01_ds 84-139 and dipeptide additives as indicated. Values are average of triplicate measurements.

FIG. 8: Melting points of HLA-A*02:01_ds 22-71 measured in TSA with addition of 100 mM final concentration of buffers at the pH indicated below the graph. Parallel experiments with and without the addition of 50 mM valeric acid are shown.

TABLE 1
Amino acid sequences.
List of Sequences

| Sequence ID: | Amino acid sequence |
|---|---|
| SEQ ID NO: 1: HLA- | GSHSMRYFFT SVSRPGRGEP RFIAVGYVDD TQFVRFDSDA |
| A*02:01_ds 84_139 | ASQRMEPRAP WIEQEGPEYW DGETRKVKAH SQTHRVDLGT |
| protein | LRGCYNQSEA GSHTVQRMYG CDVGSDWRFL RGYHQYAYDG |
| | KDYIALKEDL RSWTAADMCA QTTKHKWEAA HVAEQLRAYL |
| | EGTCVEWLRR YLENGKETLQ RTDAPKTHMT HHAVSDHEAT |
| | LRCWALSFYP AEITLTWQRD GEDQTQDTEL VETRPAGDGT |
| | FQKWAAVVVP SGQEQRYTCH VQHEGLPKPL TLRWE |
| SEQ ID NO: 2: HLA- | GSHSMRYFFT SVSRPGRGEP RCIAVGYVDD TQFVRFDSDA |
| A*02:01_ds 22_71 | ASQRMEPRAP WIEQEGPEYW DGETRKVKAH CQTHRVDLGT |
| protein | LRGYYNQSEA GSHTVQRMYG CDVGSDWRFL RGYHQYAYDG |
| | KDYIALKEDL RSWTAADMAA QTTKHKWEAA HVAEQLRAYL |
| | EGTCVEWLRR YLENGKETLQ RTDAPKTHMT HHAVSDHEAT |
| | LRCWALSFYP AEITLTWQRD GEDQTQDTEL VETRPAGDGT |
| | FQKWAAVVVP SGQEQRYTCH VQHEGLPKPL TLRWE |

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements.

Furthermore, any permutations and combinations of all described elements in this application should be considered disclosed by the description of the present application unless the context indicates otherwise.

### Definitions

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

In the following, some definitions of terms frequently used in this application are provided. These terms will, in each instance of its use, in the remainder of the application have the respectively defined meaning and preferred meanings.

As used in this application and the appended claims, the singular forms “a”, “an”, and “the” include plural referents, unless the content clearly dictates otherwise.

The term “about” refers in the context of this invention and when used in reference to a particular recited numerical value, to a value and means that the value may vary from the recited value by no more than 10%, no more than 9.5%, 9.0%. 8.5%, 8.0%, 7.5%, 7.0%, 6.5%, 6.0%, no more than 5%, no more than 4.5%, 4.0%, 3.5%, 3.0%, 2.5%, 2.0%, 1.5%, 1.0% or 0.5%. For example, as used herein, the expression “about 100” includes 95 and 105 and all values in between (e.g. 95.0, 95.5, 96.0, 96.5, 97.0, 97.5, 98.0, 98.5, 99.0, 99.5, 100.5, 101.0, 101.5, 102.0, 102.5, 103.0, 103.5, 104.0, 104.5 and 105.0).

The terms “major histocompatibility complex” or “MHC protein” or “MHC molecule” or “MHC” are used interchangeably in the context of the present invention and refer to a set of cell surface proteins, i.e. cell surface receptors, that have an essential role in establishing acquired immunity against altered natural or foreign proteins in vertebrates, which in turn determines histocompatibility within a tissue. The main function of MHC molecules is to bind to antigens derived from altered proteins or pathogens and display them on the cell surface for recognition by appropriate T-cells. The human MHC is also called HLA (human leukocyte antigen) complex or HLA. The MHC gene family is divided into three subgroups: class I, class II and class III. Complexes of peptide and MHC class I are recognized by CD8-positive T-cells bearing the appropriate TCR, whereas complexes of peptide and MHC class II molecules are recognized by CD4-positive-helper-T-cells bearing the appropriate TCR. Both types of response, CD8 and CD4 dependent, contribute jointly and synergistically to the T cell dependent immunity, e.g. an anti-tumor effect, and thus, the identification and characterization of T cells binding to particular MCH-peptide complexes or to TCRs isolated or derived therefrom is important in the development of T cell- or TCR-based immunotherapies. The term “MHC protein” as used in the context of the present invention refers both to full length MHCs as well as peptide binding fragments thereof.

The term “MHC-I” refers in the context of the present invention to MHC class I molecules or MHC-I proteins and peptide binding fragments thereof. The MHC I molecule consists of an alpha chain, also referred to as MHC I heavy chain and a beta chain, which constitutes a beta 2 microglobulin molecule. The alpha chain, comprises three alpha domains, i.e. alpha1 domain, alpha2 domain and alpha3 domain. Alpha1 and alpha2 domain mainly contribute to forming the peptide pocket to produce a peptide ligand MHC (pMHC) complex. MHC-I typically bind peptides that are derived from cytosolic antigenic proteins and which are degraded by the proteasome after ubiquitinylation and subsequently transported through a specific transporter associated with antigen processing (TAP) from the cytosol to the endoplasmic reticulum (ER). MHC I typically binds peptides of 8-12 amino acids in length.

The term “MHC-II” refers in the context of the present invention to MHC class II molecules or MHC-II proteins and peptide binding fragments thereof. The MHC-II molecule consists of an alpha chain and a beta chain, wherein the alpha chain comprises two alpha domains, alpha1 domain, alpha2 domain and the beta chain comprises two beta domains, beta1 domain and beta2 domain. MHC II typically fold in the ER in complex with a protein called invariant chain and are then transported to late endosomal compartments, where the invariant chain is cleaved by cathepsin proteases and a short fragment remains bound to the peptide-binding groove of MHC II, termed class II-associated invariant chain peptide (CLIP). This placeholder peptide is then normally exchanged against higher affinity peptides, which are derived from proteolytically degraded proteins available in endocytic compartments. MHC-II typically binds peptides of 10-30 amino acids in length or peptides of 13-25 amino acids in length.

The term “HLA” refers in the context of the present invention to human MHC molecules which differ between different human beings in amino acid sequences. However, HLAs can be identified by an internationally agreed nomenclature, the IMGT nomenclature, of HLA. The categorization to, e.g. HLA-A, is based on the identity of a given HLA to official reference sequences of each HLA, that were produced by sequence alignments. The official HLA reference sequences as well as information to the categorization system are available: www.ebi.ac.uk/ipd/imgt/hla/nomenclature/alignments.html. The website provides the following information regarding how to categorize any given HLA sequence: “The alignment files produced use the following nomenclature and numbering conventions. These conventions are based on the recommendations published for Human Gene Mutations. These were prepared by a nomenclature-working group looking at how to name and store sequences for human allelic variants. These recommendations can be found in Antonarakis SE and the Nomenclature Working Group Recommendations for a Nomenclature System for Human Gene Mutations Human Mutation (1998) 11 1-3”. Thus, the term HLA as used in the context of the present invention refers to all naturally occurring and modified HLA proteins. The HLA-A gene is located on the short arm of chromosome 6 and encodes the larger, α-chain, constituent of HLA-A. Variation of the HLA-A α-chain is key to HLA function. This variation promotes genetic diversity in the population. Since each HLA has a different affinity for peptides of certain structures, greater variety of HLAs means greater variety of antigens to be “presented” on the cell surface. Each individual can express up to two types of HLA-A, one from each of their parents. Some individuals will inherit the same HLA-A from both parents, decreasing their individual HLA diversity. However, the majority of individuals receive two different copies of HLA-A. The same pattern follows for all HLA groups. In other words, every single person can only express either one or two of the 2432 known HLA-A alleles coding for currently 1740 active proteins. HLA-A*02 signifies a specific HLA allele, wherein the letter A signifies to which HLA gene the allele belongs to and the prefix “*02 prefix” indicates the A2 serotype. In MHC class I dependent immune reactions, peptides not only have to be able to bind to certain MHC class I molecules expressed by target cells, e.g. tumor cells, they subsequently also have to be recognized by T-cells bearing specific TCRs. In the MHC class I dependent immune reaction, peptides not only have to be able to bind to certain MHC class I molecules expressed by tumor cells, they subsequently also have to be recognized by T cells bearing specific T cell receptors (TCR).

The term “peptide binding fragment of an MHC protein” as used in the context of the present invention is a part of an MHC protein or HLA protein or HLA molecule which is shorter in its amino acid length of the MHC molecule or HLA molecule and retains the biological activity of the MHC protein or HLA molecule to bind a peptide. The peptide binding fragment of an MHC protein typically comprises N- and/or C-terminal deletions, preferably C-terminal deletions that delete all or part of the transmembrane domain of the MHC. The latter is also referred to as “a soluble MHC protein” because it will not be inserted into the membrane of a cell upon expression. Peptide binding fragments of an MHC protein have under the same condition the same or essentially the same ability to bind a respective peptide as the MHC protein from which they originate. Alternatively, or additionally, a peptide binding fragment of an MHC protein can comprise the amino acid sequence of the respective MHC protein with one or more non-conservative amino acid substitution. In this case, it is preferable that the non-conservative amino acid substitution(s) do(es) not interfere with or inhibit the ability of the peptide binding fragment of an MHC protein to bind to peptides.

The term “peptide” refers in the context of this invention to a polypeptide of 2 to 26 amino acids in lengths. Peptides that are to be bound or are bound by MHC proteins to form a functional peptide-MHC complex are typically between 8 to 12 amino acids in length (also referred to as “MHC-I peptide”), if the MHC is of type I, and 13 to 25 amino acids in length, if the MHC is of type II (also referred to as “MHC-II peptide”). Peptides of shorter length, e.g. between 2 to 7 amino acids, may also be bound to folded MHC-I or MHC-II and are readily replaced, if a pre-loaded MHC I or II is contacted with a peptide of the proper length, i.e. a MHC-I peptide or MHC-II peptide (these shorter peptides are also referred to as “loading peptide”. A loading peptide may comprise at least two amino acids and does not comprise more than seven amino acids. Exemplary loading peptides are glycine-methionine (GM), glycine-tyrosine (GY), glycine-leucine (GL) or glycine-phenylalanine (GF). Whether a peptide binds to an MHC-I or MHC-II molecule depends on the peptide's natural origin, i.e. whether it is synthesized in the cytoplasm and processed in the proteasome or absorbed by endocytosis and subsequently processed. Moreover, it depends on the length of the peptide whether it will bind to the binding groove of a MHC-I or a MHC-II molecule.

The term “loading” used in the context of the present invention refers to the contacting of a loading peptide, an MHC-I peptide or MHC-II peptide with an MHC protein allowing the peptide to bind to the MHC protein. Binding of the peptide to the MHC protein means binding by non-covalent bonds, including the formation of hydrogen bonds.

The term “hydrocarbyl” as used in the context of the present invention refers to an organic molecule that merely consists of carbon and hydrogen atoms and the “-yl” in the term “hydrocarbyl” refers to a univalent group consisting only of carbon and hydrogen atoms from which one hydrogen has been removed and replaced with another group. Such a group is also referred to as “radical”. An example for a hydrocarbyl is an alkyl, such as ethyl, —CH₂—CH₃.

The hydrocarbyl may be branched or unbranched and may be saturated or unsaturated, i.e. branched saturated, branched unsaturated, unbranched saturated and unbranched unsaturated. The hydrocarbyl may be unsubstituted or substituted. In the latter case, atoms other than carbon and hydrogen are covalently linked to one or more of the carbon atoms of the hydrocarbyl. Preferred atoms that may be covalently linked to the carbon atoms are halogen, in particular F, Cl, or Br; —OH; —O—R₄; ═O; —NO₂; —NR₂R₃, wherein R₂ and R₃ are independently selected from H, and hydrocarbyl; and —N—R₄, wherein R₄ is selected from H and hydrocarbyl.

The term “salt” as used in the context of the present invention relates to an ionic salt of the hydrocarbyl-COOH, in particular to an ionic salt of the carboxylic acid, which is formed by the reaction of a carboxylic acid with a base and comprises a cation and the negatively charged carboxy group as an anion. Typical cations that are comprised in ionic salts of carboxylic acids are lithium, sodium, potassium, rubidium, caesium or francium, magnesium, calcium or aluminium.

The term “buffer substance” as used in the context of the present invention relates to a weak acid or base used to maintain the acidity, i.e. the pH value of a solution, a suspension and/or an emulsion near a selected value after the addition of another acid or base. The function of a buffer substance is to prevent a rapid change in the pH value when acids or bases are added to the solution, suspension or emulsion. In an aqueous solution, suspension and/or emulsion, a buffer is present in a mixture of a weak acid and its conjugate base or a in a mixture of a weak base and its conjugated acid. Examples of buffer substances include, but are not limited to the following: sodium bicarbonate; acetic acid or acetate salts (e.g. sodium acetate, zinc acetate); boric acid or boric salts; N-cyclohexyl-2-aminoethanesulfonic acid (CHES) or salts thereof; 3-[[1,3-dihydroxy-2-(hydroxymethyl)propan-2-yl]amino]propane-1-sulfonic acid (TAPS) or salts thereof; 2-(N-morpholino)ethanesulfonic acid (MES) and salts thereof; piperazine-N,N′-bis(2-ethanesulfonic acid (PIPES) and salts thereof; N-(2-acetamido)-2-aminoethane-sulfonic acid (ACES) and salts thereof; N-(2-acetamido)iminodiacetic acid, N-(carbamoylmethyl)iminodiacetic acid (ADA) and salts thereof; cholamine chloride; N,N-bis[2-Hydroxyethyl]-2-aminoethanesulfonic acid (BES) and salts thereof; Bis(2-hydroxyethyl)amino-tris(hydroxymethyl)methan (Bis Tris) and salts thereof; 2,2′-(Propane-1,3-diyldiimino)bis[2-(hydroxymethyl)propane-1,3-diol] (Bis-Tris propane) and salts thereof; —N-Bis-(hydroxyethyl)-amino-2-hydroxy-propansulfonsaure (DIPSO) and salts thereof; N-2-Hydroxyethylpiperazin-N′-3-propansulfonsaure (HEPPS or EPPS) and salts thereof; N-(Hydroxyethyl)piperazine-N′-2-hydroxypropanesulfonic acid (HEPPSO) and salts thereof; [2-[[1,3-dihydroxy-2-(hydroxymethyl)-propan-2-yl]amino]ethanesulfonic acid (TES) and salts thereof; 2 2-[4-(2-Hydroxyethyl)piperazin-1-yl]ethane-1-sulfonic acid (HEPES) and salts thereof; acetamidoglycine; N-[2-hydroxy-1, 1-bis(hydroxylmethyl) ethyl]glycine (tricine); glycinamide; 2-(bis(2-hydroxyethyl)amino)acetic acid (bicine) and salts thereof; propionate salts; 3-[[1,3-dihydroxy-2-(hydroxymethyl)propan-2-yl]-amino]-2-hydroxypropane-1-sulfonic acid (TAPSO) and salts thereof; 2,2′,2″-Trihydroxytriethylamine (TEA) and salts thereof; 3-morpholinopropane-1-sulfonic acid (MOPS) and salts thereof; 2-Hydroxy-3-(morpholin-4-yl)propane-1-sulfonic acid (MOPSO) and salts thereof; Piperazine-N,N′-bis(2-hydroxypropanesulfonic acid) (POPSO) and salts thereof; saline-sodium citrate (SSC) buffer; 2-amino-2-hydroxymethyl-propane-1,3-diol (synonyms: TRIS, trisamine, THAM, tromethamine, trometamol, tromethane); imidazole, citric acid or citrate salts, for example sodium citrate); trisodium phosphate, disodium hydrogen phosphate, sodium dihydrogen phosphate, tripotassium phosphate, dipotassium phosphate, monopotassium phosphate and/or any other buffer substance containing phosphate, e.g. phosphate buffered saline (PBS). Amino acids (having free basic or acidic functional groups, e.g. methionine, arginine), peptides or proteins (having free basic or acidic functional groups) may also be used as buffer substance.

### EMBODIMENTS

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect the invention relates to a composition comprising
(i) a major histocompatibility complex (MHC) protein; and
(ii) an R₁—COOH or salt thereof;
wherein R₁ is substituted or unsubstituted hydrocarbyl.

In one embodiment of the first aspect of the present invention the composition comprises an MHC protein and an R₁—COOH, i.e. a hydrocarbyl residue substituted with a carboxyl group and wherein R₁ is unsubstituted. In one embodiment the composition comprises an MHC protein and an R₁—COOH, i.e. a hydrocarbyl residue substituted with a carboxyl group and wherein R₁ is substituted. In one embodiment the composition comprises an MHC protein, a R₁—COOH, wherein R₁ is substituted, and a loading peptide, MHC-I peptide and/or MHC-II peptide, which is bound to said MHC protein. In one embodiment the composition comprises an MHC protein, an R₁—COOH, wherein R₁ is unsubstituted and a loading peptide, MHC-I peptide and/or MHC-II peptide which is bound to said MHC.

The molecule “R₁—COOH” comprises a hydrocarbyl residue (R₁) which is substituted with a carboxylic acid moiety or group, i.e. one hydrogen atom of the hydrocarbyl is replaced with a carboxyl group, i.e. —COOH. Thus, a hydrocarbon group is univalently attached to a carboxyl group. In one embodiment R₁ may comprises saturated branched and linear carbohydrates, i.e. alkyl groups, as well as branched and linear unsaturated carbohydrates, including alkenyl or alkynyl groups, attached to the carboxy group. R₁ may be further substituted at one or more carbon atoms of the hydrocarbyl by removing a hydrogen atom. If R₁ is substituted, it is preferred that R₁ is substituted with a halogen, in particular F, Cl, Br, or I; —OH; —O—R₄; ═O; —NO₂; —NR₂R₃, wherein R₂ and R₃ are independently selected from H, and hydrocarbyl; and —N—R₄, wherein R₄ is selected from H and hydrocarbyl. Particularly suitable substituents are halogens, e.g. F, Cl or Br. The R₁—COOH may also be a salt, i.e. an ionic salt of the carboxylic acid group R₁—COO⁻ Me⁺, wherein Me⁺ is a cation, in particular an alkali metal ion, an alkaline earth metal ion, NH₄⁺ or a metal ion from the boron group. In one embodiment the composition comprises an MHC protein and a salt of R₁—COOH selected from the group consisting of lithium, sodium, potassium, magnesium, calcium, ammonium or aluminium. More preferably the salt of R₁—COOH is a sodium salt.

In one embodiment of the first aspect of the present invention the composition comprises an MHC protein and an R₁—COOH, wherein R₁ is C₂-C₂₁-hydrocarbyl. In one embodiment R₁ is linear C₂-C₂₁-alkyl. In another embodiment R₁ is branched C₃₋₂₁-alkyl. In another embodiment R₁ is linear C₂₋₂₁-alkenyl. In another embodiment R₁ is branched C₃₋₂₁-alkenyl. In another embodiment R₁ is linear C₂₋₂₁-alkynyl. In another embodiment R₁ is branched C₄₋₂₁-alkynyl. It is preferred that R₁ is linear C₂-C₂₁-alkyl. In another preferred embodiment R₁ is branched C₃₋₂₁-alkyl. In another preferred embodiment R₁ is linear C₂₋₂₁-alkenyl. In another preferred embodiment R₁ is branched C₃₋₂₁-alkenyl.

In one embodiment of the first aspect of the present invention the composition comprises an MHC protein and an R₁—COOH, wherein R₁ is linear C₂₋₂₁-alkyl, preferably R₁ is linear C₂₋₈-alkyl or C₂₋₇-alkyl, more preferably R₁ is linear C₂₋₇-alkyl or R₁ is C₃₋₇-alkyl and even more preferably R₁ is linear C₄₋₆-alkyl. In another embodiment the composition comprises an MHC protein and R₁—COOH wherein R₁ is linear C₂₋₂₁-alkyl, which is preferably selected from the group consisting of ethyl, propyl, butyl, pentyl, hexyl, heptyl or octyl. More preferably R₁ is linear C₂₋₂₁-alkyl selected from the group consisting of butyl, pentyl or hexyl. Even more preferably the composition comprises an MHC and a R₁—COOH wherein R₁ is linear C₂₋₂₁-alkyl which is pentyl. In each case the linear C₂₋₂₁-alkyl, preferably linear C₂₋₈-alkyl or C₂₋₇-alkyl, more preferably linear C₂₋₇-alkyl or C₃₋₇-alkyl and even more preferably linear C₄₋₆-alkyl, may be unsubstituted or substituted, preferably unsubstituted.

In another embodiment the composition comprises an MHC protein and an R₁—COOH, wherein R₁ is branched C₃₋₂₁-alkyl, preferably R₁ is branched C₃₋₈-alkyl, more preferably R₁ is branched C₃₋₇-alkyl, more preferably R₁ is branched C₄₋₆-alkyl. In one embodiment R₁ is selected from the group consisting of iso-propyl, iso-butyl, sec-butyl, tert-butyl, 1-methyl-butyl, 2-methyl-butyl, 3-methyl-butyl, 1,1-dimethyl-propyl, 2,2-dimethyl-propyl, 1-ethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2, trimethyl-propyl, 1-ethyl-2-methyl-propyl, 1-methyl-hexyl, 2-methyl-hexyl, 3-methyl-hexyl, 4-methyl-hexyl, 5-methyl-hexyl, 1,1-dimethyl-pentyl, 1,2-dimethyl-pentyl, 1,3-dimethyl-pentyl, 1,4-dimethyl-pentyl, 2,2-dimethyl-pentyl, 2,3-dimethyl-pentyl, 2,4-dimethyl-pentyl, 3,3-dimethyl-pentyl, 3,4-dimethyl-pentyl, 1-ethyl-pentyl, 2-ethyl-pentyl, 3-ethyl-pentyl, 1,1,2-trimethyl-butyl, 1,1,3-trimethyl-butyl, 1,2,3-trimethyl-butyl, 1,2,2-trimethyl-butyl, 2,2,3-trimethyl-butyl, 1-methyl-1-ethyl-butyl, 1-ethyl-2-methyl-butyl, 1-ethyl-3-methyl-butyl, 1-methyl-2-ethyl-butyl, 2-methyl-2-ethyl-butyl, 1-propy-butyl, 1-methyl-heptyl, 2-methyl-heptyl, 3-methyl-heptyl, 4-methyl-heptyl, 5-methyl-heptyl, 6-methyl-heptyl, 1,1-dimethyl-hexyl, 1,2-dimethyl-hexyl, 1,3-dimethyl-hexyl, 1,4-dimethyl-hexyl, 1,5-dimethyl-hexyl, 2,2-dimethyl-hexyl, 2,3-dimethyl-hexyl, 2,4-dimethyl-hexyl, 2,5-dimethyl-hexyl, 3,3-dimethyl-hexyl, 3,4-dimethyl-hexyl, 3,5-dimethyl-hexyl, 4,4-dimethyl-hexyl, 4,5-dimethyl-hexyl, 5,5-dimethyl-hexyl, 1-ethyl-hexyl, 2-ethyl-hexyl, 3-ethyl-hexyl or 4-ethyl-hexyl. In each case the branched C₃₋₂₁-alkyl, preferably branched C₃₋₇-alkyl, more preferably branched C₄₋₆-alkyl may be unsubstituted or substituted, preferably unsubstituted. It is particularly preferred that R₁ is branched C₄-alkyl or branched C₅-alkyl.

In another embodiment the composition comprises an MHC protein and an R₁—COOH, wherein R₁ is linear C₂₋₂₁-alkenyl, preferably R₁ is linear C₂₋₈-alkenyl or linear C₂₋₇, more preferably R₁ is linear C₃₋₇-alkenyl and most preferably R₁ is linear C₄₋₆-alkenyl. In one embodiment R₁ is selected from the group consisting of ethenyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 1-heptenyl, 4-heptenyl, 5-heptenyl, 5-heptenyl, 6-heptenyl, 1-octenyl, 2-octenyl, 3-octenyl, 4-octenyl, 5-octenyl, 6-octenyl or 7-octenyl. In each case the linear C₂₋₂₁-alkenyl, preferably linear C₂₋₈-alkenyl or linear C₂₋₇, more preferably linear C₃₋₇-alkenyl and most preferably C₄₋₆-alkenyl may be unsubstituted or substituted, preferably unsubstituted.

In another embodiment the composition comprises an MHC protein and an R₁—COOH, wherein R₁ is branched C₃₋₂₁-alkenyl, preferably R₁ is branched C₃₋₈-alkenyl, more preferably R₁ is branched C₃₋₇-alkenyl and most preferably R₁ is branched C₄₋₆-alkenyl. In one embodiment R₁ is selected from the group consisting of 1-methyl-ethenyl, 1-methyl-1-propenyl, 2-methyl-1-propenyl, 1-methyl-2-propenyl, 2-methyl-2-propenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-Methyl-4-pentenyl, 2-methyl-4-pentenyl, 3-methyl-4-pentenyl, 4-methyl-4-pentenyl, 1,1-dimethyl-2-butenyl, 1,1-Dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-Dimethyl-2-butenyl, 2,3-dimethyl-3-butenyl, 3,3-dimethyl-1-butenyl, 3,3-dimethyl-2-butenyl, 1-ethyl-1-butenyl, 1-Ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, or 1-ethyl-2-methyl-2-propenyl. In each case the branched C₃₋₂₁-alkenyl, preferably branched C₃₋₈-alkenyl, more preferably branched C₃₋₇-alkenyl and most preferably branched C₄₋₆-alkenyl may be unsubstituted or substituted, preferably unsubstituted.

In one embodiment the composition comprises an MHC protein and an R₁—COOH, wherein R₁ is linear C₂₋₂₁-alkynyl, preferably R₁ is linear C₂₋₈-alkynyl or linear C₂₋₇-alkynyl, more preferably R₁ is linear C₃₋₇-alkynyl, and most preferably R₁ is linear C₄₋₆-alkynyl. In one embodiment R₁ is selected from the group consisting of ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 2-heptynyl, 1-heptynyl, 4-heptynyl, 5-heptynyl, 5-heptynyl, 6-heptynyl, 1-octynyl, 2-octynyl, 3-octynyl, 4-octenyl, 5-octynyl, 6-octynyl or 7-octynyl. In each case the linear C₂₋₂₁-alkynyl, preferably linear C₂₋₈-alkynyl or linear C₂₋₇-alkynyl, more preferably linear C₃₋₇-alkynyl, and most preferably linear C₄₋₆-alkynyl may be unsubstituted or substituted, preferably unsubstituted.

In one embodiment the composition comprises an MHC protein and an R₁—COOH, wherein R₁ is branched C₄₋₂₁-alkynyl, preferably R₁ is branched C₄₋₈-alkynyl, more preferably R₁ is branched C₄₋₇-alkynyl, and most preferably R₁ is branched C₄₋₆-alkynyl. In one embodiment R₁ is selected from the group consisting of 1-methyl-2-propynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl 1-ethyl-3-butynyl, 2-ethyl-3-butynyl or 1-ethyl-1-methyl-2-propynyl. In each case the branched C₄₋₂₁-alkynyl, preferably linear C₄₋₈-alkynyl, more preferably branched C₄₋₇-alkynyl, and most preferably branched C₄₋₆-alkynyl may be unsubstituted or substituted, preferably unsubstituted.

In another embodiment of the first aspect of the present invention the composition comprises an MHC protein and an R₁—COOH, wherein R₁ is hydrocarbyl and wherein the double or triple bond is between the C₁ and C₂ atom or the double or triple bond is between the C₂ and C₃ atom of the hydrocarbyl (the numbering is with respect to the carbon of the hydrocarbyl attached to the COOH group).

Preferred examples are linear C₂₋₂₁-1-alkenyl, preferably linear C₂₋₈-1-alkenyl or linear C₂₋₇-alkenyl, more preferably linear C₃₋₇-1-alkenyl and most preferably C₄₋₆-1-alkenyl; or C₂₋₂₁-2-alkenyl, preferably linear C₂₋₈-2-alkenyl or linear C₂₋₇-2-alkenyl, more preferably linear C₃₋₇-2-alkenyl and most preferably linear C₄₋₆-2-alkenyl.

Preferred examples are branched C₃₋₂₁-1-alkenyl, preferably branched C₃₋₈-1-alkenyl, more preferably branched C₃₋₇-1-alkenyl and most preferably branched C₄₋₆-1-alkenyl; or branched C₃₋₂₁-2-alkenyl, preferably branched C₃₋₈-2-alkenyl, more preferably branched C₃₋₇-2-alkenyl and most preferably branched C₄₋₆-2-alkenyl.

Preferred examples are linear C₂₋₂₁-1-alkynyl, preferably linear C₂₋₈-1-alkynyl or linear C₂₋₇-1-alkynyl, more preferably linear C₃₋₇-1-alkynyl, and most preferably linear C₄₋₆-1-alkynyl; or linear C₂₋₂₁-2-alkynyl, preferably linear C₂₋₈-2-alkynyl or C₂₋₇-2-alkynyl, more preferably linear C₃₋₇-2-alkynyl, and most preferably linear C₄₋₆-2-alkynyl.

Preferred examples are branched C₃₋₂₁-1-alkynyl, preferably branched C₃₋₈-1-alkynyl, more preferably branched C₃₋₇-1-alkynyl, and most preferably branched C₄₋₆-1-alkynyl; or branched C₃₋₂₁-2-alkynyl, preferably branched C₃₋₈-2-alkynyl, more preferably branched C₃₋₇-2-alkynyl, and most preferably branched C₄₋₆-2-alkynyl.

Thus, particularly preferred linear alkenyls are 1-propenyl, 2-propenyl, 1-butenyl, 2-butenyl, 1-pentenyl, 2-pentenyl, 1-hexenyl, 2-hexenyl, 1-heptenyl, 2-heptenyl, 1-octenyl, 2-octenyl, 1-noneyl, and 2-nonenyl. Accordingly, preferred branched alkenyls comprise 1-propenyl, 2-propenyl, 1-butenyl, 2-butenyl, 1-pentenyl, 2-pentenyl, 1-hexenyl, 2-hexenyl, 1-heptenyl, 2-heptenyl, 1-octenyl, and 2-octenyl as base chain and a methyl, ethyl or propyl branch. Thus, preferred linear alkynyls are 1-propynyl, 2-propynyl, 1-butynyl, 2-butynyl, 1-pentynyl, 2-pentynyl, 1-hexynyl, 2-hexynyl, 1-heptynyl, 2-heptynyl, 1-octynyl, 2-octynyl, 1-nonynyl, and 2-nonynyl. Accordingly, preferred branched alkynyls comprise 1-butynyl, 2-butynyl, 1-pentynyl, 2-pentynyl, 1-hexynyl, 2-hexynyl, 1-heptynyl, 2-heptynyl, 1-octynyl and 2-octynyl as base chain and a methyl, ethyl or propyl branch. In each case the linear alkynyl, preferably linear C₂₋₈-alkynyl linear C₂₋₇-alkynyl, more preferably linear C₃₋₇-alkynyl, and most preferably linear C₄₋₆-alkynyl may be unsubstituted or substituted, preferably unsubstituted.

In one embodiment the composition comprises an MHC protein and an R₁—COOH, wherein R₁ is hydrocarbyl and the hydrocarbyl is substituted with one or more substituents selected from a halogen, in particular F, Cl, Br, or I; —OH; —O—R₄; ═O; —NO₂; —NR₂R₃, wherein R₂ and R₃ are independently selected from H, and hydrocarbyl; and —NH—R₄, wherein R₄ is selected from H and hydrocarbyl. Particular suitable substituents are halogens, e.g. F, Cl or Br. In one embodiment the composition comprises an MHC protein and a R₁—COOH, wherein R₁ is hydrocarbyl and wherein the double or triple bond is between the C₁ and C₂ atom or the C₂ and C₃ atom of the hydrocarbyl and the hydrocarbyl is substituted with one or more substituents selected from a halogen, in particular F, Cl, Br, or I; —OH; —O—R₄; ═O; —NO₂; —NR₂R₃, wherein R₂ and R₃ are independently selected from H, and hydrocarbyl; and —NH—R₄, wherein R₄ is selected from H and hydrocarbyl. Particular suitable substituents are halogens, e.g. F, Cl or Br. In one preferred embodiment if R₂ and R₃ are H then —NR₂R₃ is not attached to the C₁ carbon atom (the numbering of the carbon atoms in the hydrocarbyl is with respect to the first carbon of the hydrocarbyl attached to the COOH group), i.e. it is preferred that R₁—COOH is not an α-amino acid. In one preferred embodiment the hydrocarbyl is substituted with one or more substituents selected from a halogen, in particular F, Cl, Br, or I; —OH; —O—R₄; ═O; —NO₂; —NR₂R₃, wherein R₂ and R₃ are independently selected from hydrocarbyl; and —NH—R₄, wherein R₄ is hydrocarbyl. In one preferred embodiment the hydrocarbyl is substituted with one or more substituents selected from a halogen, in particular F, Cl, Br, or I; —OH; —O—R₄; ═O; —NO₂; —NR₂R₃, wherein R₂ and R₃ are independently selected from hydrocarbyl; and —NH—R₄, wherein R₄ is hydrocarbyl. Preferably, R₂, R₃ and R₄ are independent of each other selected from C₁₋₈-alkyl.

In one preferred embodiment R₁—COOH or salt thereof is a mono-carboxylic acid.

In a preferred embodiment of the first aspect of the present invention, the composition comprises an MHC protein and an R₁—COOH, wherein R₁—COOH is a fatty acid, preferably a naturally occurring fatty acid. More preferably, the fatty acid comprises 3-9 carbon atoms. Even more preferably, the fatty acid comprises 3-8 or 4-8 carbon atoms. Most preferably, the fatty acid comprises 5-7 carbon atoms.

In another embodiment of the first aspect of the present invention the composition comprises an MHC protein and an R₁—COOH, wherein R₁—COOH is a C₂₋₂₁-alkyl-COOH, selected from the group consisting of C₃-C₂₁-alkyl, preferably of propanoic acid, n-butanoic acid, iso-butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid or docosanoic acid. In a preferred embodiment the composition comprises an MHC protein and a C₂₋₂₁-alkyl-COOH selected from the group consisting of propanoic acid, butanoic acid, pentanoic acid, hexanoic acid, heptanoic acid, octanoic acid and nonanoic acid. In an even more preferred embodiment, the composition comprises an MHC protein and a C₂₋₂₁-alkyl-COOH selected from the group consisting of propanoic acid, butanoic acid, pentanoic acid, hexanoic acid, heptanoic acid, and octanoic acid. In an even more preferred embodiment, the composition comprises an MHC protein and a pentanoic acid, hexanoic acid or heptanoic acid.

In another embodiment of the first aspect of the present invention the composition comprises an MHC protein and an R₁—COOH, wherein R₁—COOH is branched C₃₋₂₁-alkyl-COOH selected from the group consisting of isobutanoic acid, isopentanoic acid, 3-methyl-pentanoic acid and 4-methyl-pentanoic acid. In a preferred embodiment, the composition comprises an MHC protein and an R₁—COOH, wherein R₁—COOH is branched C₃₋₂₁-alkyl-COOH selected from the group consisting of isobutanoic acid, isopentanoic acid and 4-methyl-pentanoic acid. In an even more preferred embodiment, the composition comprises an MHC protein and R₁—COOH, wherein R₁—COOH is isopentanoic acid and 4-methyl-pentanoic acid.

In one embodiment the composition comprises an MHC protein and an R₁—COOH, wherein R₁—COOH is C₂₋₂₁-alkenyl-COOH selected from the group consisting of 2-hexenoic acid, 3-hexenoic acid and 5-hexenoic acid. In a preferred embodiment the composition comprises an MHC protein and C₂₋₂₁-alkenyl-COOH, wherein C₂₋₂₁-alkenyl-COOH is 2-hexenoic acid or 3-hexenoic acid.

Table 2 below comprises synonyms of selected compounds:

TABLE 2
Selected compound IUPAC names and synonyms.

| IUPAC name: | Synonyms: |
|---|---|
| Propanoic acid | Propionic acid |
| Butanoic acid | Butyric acid |
| Pentanoic acid | Valeric acid |
| | 1-Butanecarboxylic acid |
| | Propylacetic acid |
| 3-methylbutanoic acid | Delphinic acid |
| | 3-Methylbutyric acid |
| | Isopentanoic acid |
| | Isovaleric acid |
| 3-methyl-pentanoic acid | 3-methylvaleric acid |
| 4-methyl-pentanoic acid | 4-Methylvaleric acid |
| | Isocaproic acid |

It is envisioned that the molar concentration of R₁—COOH exceeds the molar concentration of the MHC protein. In another embodiment of the first aspect of the present invention the composition comprises an MHC protein and an R₁—COOH, wherein the concentration of the R₁—COOH is about 5 mM to about 100 mM, about 10 mM to about 80 mM, about 15 mM to about 70 mM, about 20 mM to about 60 mM, about 25 mM to about 50 mM, about 30 mM to about 40 mM, or about 30 mM to about 35 mM. In a preferred embodiment the composition comprises an MHC protein and an R₁—COOH, wherein the concentration of the R₁—COOH is about 5 mM to about 100 mM, about 10 mM to about 80 mM, about 15 mM to about 70 mM, about 20 mM to about 60 mM, or about 25 mM to about 50 mM. In a most preferred embodiment the composition comprises an MHC protein and an R₁—COOH, wherein the concentration of the R₁—COOH is about 50 mM.

In another embodiment the composition comprises an MHC protein and an R₁—COOH, wherein the concentration of the R₁—COOH is 5 mM to 100 mM, 10 mM to 80 mM, 15 mM to 70 mM, 20 mM to 60 mM, 25 mM to 50 mM, 30 mM to 40 mM, or 30 mM to 35 mM. In a preferred embodiment the composition comprises an MHC protein and an R₁—COOH, wherein the concentration of the R₁—COOH is 5 mM to 100 mM, 10 mM to 80 mM, 15 mM to 70 mM, 20 mM to 60 mM, or 25 mM to 50 mM. In a most preferred embodiment the composition comprises an MHC protein and an R₁—COOH, wherein the concentration of the R₁—COOH is 50 mM.

In another embodiment of the first aspect of the present invention the composition comprises an MHC protein and an R₁—COOH in a dried form, i.e. not in a solution but for example as powder, e.g. freeze-dried powder, which is ready for reconstitution prior to loading with a peptide, preferably a loading peptide, an MHC-I peptide or MHC-II peptide. In a preferred embodiment the composition comprises an MHC protein and an R₁—COOH and is a solution, preferably an aqueous solution. In a further embodiment, the composition comprises an MHC protein and an R₁—COOH that are in solution. The term “aqueous” as used in the context of the present invention refers to a solution in which the solvent is water and/or to a suspension in which the external phase is water and/or to an emulsion in which the dispersed or continuous phase is water.

In another embodiment of the first aspect of the present invention the composition comprises an MHC protein and an R₁—COOH according to any one of the above-outlined embodiments wherein the pH of the composition is in the range of about pH 6 to about pH 9, about pH 6 to about pH 8, about pH 6 to about pH 7.5, or about pH 6.4 to about pH 7.5. In another embodiment of the first aspect of the present invention the composition comprises an MHC protein and an R₁—COOH according to any one of the above-outlined embodiments, wherein the pH of the composition is in the range of pH 6 to pH 9, pH 6 to pH 8, pH 6 to pH 7.5, or pH 6.4 to pH 7.5. In a preferred embodiment the composition comprises an MHC protein and an R₁—COOH according to any one of the above-outline embodiments and the pH of the composition is in the range of pH 6.4 to pH 7.5. In an even more preferred embodiment the composition comprises an MHC protein and an R₁—COOH according to any one of the above-outlined embodiments and the pH of the composition is in the range of pH 6 to 7.2. If the composition of the MHC protein and an R₁—COOH or salt thereof does have a pH that differs from the indicated pH values, then the respectively desired pH is attained by adding an appropriate amount of a buffer in addition to the MHC protein and the R₁—COOH or salt thereof. Suitable buffer substances are well known in the art and are also described further below.

As used in the context of the present invention, the MHC protein can be bound to a peptide. Thus, the composition may comprise (i) a major histocompatibility complex (MHC) protein; (ii) an R₁—COOH or salt thereof, wherein R₁ is substituted or unsubstituted hydrocarbyl and a peptide bound to said MHC protein. Further, the kit may comprise: (i) an MHC protein; (ii) an R₁—COOH or salt thereof; and a peptide. In preferred embodiments, the MHC protein is not bound to peptide. In other words, the MHC protein is essentially free of peptide. In further preferred embodiments, the MHC protein does not comprise a peptide.

In another embodiment of the first aspect of the present invention the composition comprises an MHC protein and an R₁—COOH, wherein the composition is an aqueous solution and comprises a buffer substance selected from the group consisting of ACES, ADA, BES, Bis-Tris Propane, DIPSO, EPPS, HEPPSO, Imidazol, MOBS, MES, Bis-Tris, MOPS, TRIS, MOPSO, Phosphate, PIPES, POPSO, TAPSO, TEA, TES, Tricine or HEPES buffer. In a preferred embodiment the composition comprises an MHC and an R₁—COOH, wherein the composition is an aqueous solution and comprises as buffer substance MES, Bis-Tris, MOPS, HEPES or TRIS. In an even more preferred embodiment the composition comprises an MHC protein and an R₁—COOH, wherein the composition is an aqueous solution and comprises HEPES buffer.

In another embodiment of the first aspect of the present invention the composition comprises an MHC protein and an R₁—COOH, wherein the MHC protein comprised in the composition has an increased stability. Preferably, the composition is an aqueous solution. Compositions comprising the MHC protein stabilized by R₁—COOH are typically compared to compositions comprising said MHC protein without R₁—COOH. The term “increased stability” or “improved stability” or “stabilized” or the like refers to the ability to increase and/or improve the protein's equilibrium state, such that the native state of the protein (e.g. the three dimensional structure of the protein) is improved and/or favored. A stabilized MHC protein may show less aggregation during the protein lifetime, including during processes such as refolding, purification, sterilization, shipping or storage. Thus, a stabilized MHC protein according to the composition of the first aspect of the invention may provide a longer shelf-life. In particularly preferred embodiments, the stabilized MHC protein according to the composition of the first aspect of the invention has an increased melting temperature (Tin). Thus, in particularly preferred embodiments, the term “stabilizing an MHC protein” or grammatical variants thereof refers to an MHC protein having an increased melting temperature (Tm).

In certain embodiments, the term “stability” as used in the context of the present invention may refer to chemical and/or physical stability of proteins. Both depend inter alia on parameters such as solvent, pH value and/or other excipients or additives, such as surfactants, salts or other small molecules in the composition. The MHC protein may, in other words, be stabilized by the addition of R—COOH leading to increased chemical and/or physical stability of the MHC protein comprised in the composition.

“Physical stability” as used in the context of the present invention relates to the stability of proteins without any change in the chemical composition of the protein and includes processes such as denaturation, for example increased temperature; surface adsorption; aggregation and/or precipitation of the protein. It is envisioned in certain embodiments that the MHC protein which is stabilized by R₁—COOH shows less aggregation during the protein lifetime, including during processes such as refolding, purification, sterilization, shipping or storage. Protein aggregation can be inter alia measured by visual or microscopic inspection, for example fluorescence microscopy; ultracentrifugation; mass spectrometry; conductivity-based particle count; laser diffraction; size exclusion chromatography or electrophoresis. Therefore, the composition of the first aspect of the invention comprising an MHC protein, which is stabilized by R₁—COOH may show an increased physical stability of the MHC protein, and may show an increased half-life leading to increased storage stability. Storage stability can be assessed at 4° C., room temperature or 40° C., for example. Preferably, the MHC protein shows an increased storage stability at 4° C. “Chemical stability” of proteins as used in the context of the present invention may refer to a change of the chemical structure of the protein as a direct or indirect effect. Examples of chemical stability can be hydrolysis of a peptide bond, deamidation of amino acid residues, for example deamidation of asparagine (N) residues in solution or in solid state; deamidation of glutamine (Q) residues; succinimide formation; isomerization of amino acid residues, for example aspartate (D); hydrolysis of aspartate (D) or tryptophane (W), racemization; β-elimination and oxidation. It is envisioned in certain embodiments that the MHC protein, which is stabilized by R₁—COOH, shows increased chemical stability compared to a composition comprising said MHC protein but not the Rr-COOH.

In another embodiment, the composition of the first aspect of the invention comprising an MHC protein which is stabilized by R₁—COOH may show increased chemical stability and increased physical stability, for example, storage stability at 4° C. In another embodiment the MHC protein comprised in the composition of the first aspect of the present invention increases the melting temperature (Tm) of the MHC protein by at least 0.5° C., 1.0° C., 1.5° C., 2.0° C., 2.5° C., 3.0° C., 3.5° C., 4.0° C., 4.5° C., 5.0° C., 5.5° C. or 6.0° C. In another embodiment the MHC protein comprised in the composition of the first aspect of the present invention is stabilized by R₁—COOH, which increases the melting temperature of the MHC protein comprised in the composition by at least 0.5° C., 1.0° C., 1.5° C., 2.0° C., 2.5° C., 3.0° C., 3.5° C., 4.0° C., 4.5° C., 5.0° C., 5.5° C. or 6.0° C. Preferably, the R₁—COOH is comprised in or is added to the composition of the first aspect of the invention in a concentration that it increases the melting temperature of the MHC protein comprised in the composition by at least 0.5° C., 1.0° C., 1.5° C., 20° C., 2.5° C., 3.0° C., 3.5° C., 4.0° C., 4.5° C., 5.0° C., 5.5° C. or 6.0° C.

In another embodiment of the first aspect of the present invention the composition comprises an MHC protein and an R₁—COOH or salt thereof, wherein the composition is an aqueous solution, wherein the MHC protein comprised in the composition has an increased melting temperature (Tm), wherein the Tm of said MHC protein is increased, if the Tm of the MHC protein in a composition according to (a) is higher than the Tm of the MHC protein in a composition according to (b):
(a) an aqueous composition consisting of the MHC protein, 20 mM HEPES buffer at pH 7, and R₁—COOH or salt thereof,
(b) an aqueous composition consisting of the same MHC protein as in (a) and 20 mM HEPES buffer at pH 7.

In a preferred embodiment of the first aspect of the present invention the composition comprises an MHC protein and an R₁—COOH or salt thereof, wherein the composition is an aqueous solution and the R₁—COOH or salt thereof is comprised in the composition in an amount that the melting temperature (Tm) of MHC protein is increased and wherein the Tm of said MHC protein is increased, if the Tm of the MHC protein in a composition according to (a) is higher than the Tm of the MHC protein in a composition according to (b):
(a) an aqueous composition consisting of the MHC protein, 20 mM HEPES buffer at pH 7, and R₁—COOH or salt thereof,
(b) an aqueous composition consisting of the same MHC protein as in (a) and 20 mM HEPES buffer at pH 7.

The MHC protein in both compositions (a) and (b) is not bound to a peptide, e.g. the dipeptide. Thus, the MHC can be essentially peptide-free. “Essentially peptide-free” as used in the context of the present invention means that if—at all—only residual amounts of peptide is bound to the MHC protein. Essentially peptide-free MHC proteins can be obtained by removing the peptide (e.g. the dipeptide), for example by size exclusion chromatography as shown in the Examples. The Tm can also be determined by the methods provided in below Examples. For example, the increase of Tm can be determined in the Applied Biosystems 7500 real time PCR instrument at a protein concentration of 0.1 mg/ml in a buffer of 20 mM Tris pH 8.0 or another buffer, for example 20 mM HEPES buffer at pH 7, 150 mM NaCl and the R₁—COOH or salt thereof, compared to the same buffer comprising water instead of R₁—COOH or salt thereof. In this method, no additive, such as R₁—COOH, fatty acids, glycerol, peptides etc., stabilizing the MHC is present in the solution. The following Tm increases may be determined by the above described methods. It is herein preferred that the melting temperature of the composition is increased by at least 0.5° C., 1.0° C., 1.5° C., 2.0° C., 2.5° C., 3.0° C., 3.5° C., 4.0° C., 4.5° C., 5.0° C., 5.5° C. or 6.0° C. In even more preferred embodiments, the melting temperature of the composition is increased by at least 0.5° C., 1.0° C., 1.5° C., 2.0° C., 2.5° C., 3.0° C., 3.5° C., 4.0° C., 4.5° C., or 5.0° C. The increase of melting temperature is typically determined by comparison of two samples, for example the Tm is determined in a sample comprising the composition comprising inter alia an MHC protein and a R₁—COOH or salt thereof and is compared to the Tm determined in a sample comprising a composition inter alia comprising an MHC protein, preferably the identical MHC protein, but without a R₁—COOH or salt thereof; or comprising a composition according to (b) described above. The term “melting temperature (Tm)” refers in the context of the present invention to the temperature at which the maximal rate of protein unfolding is observed in a temperature gradient. An exemplary way how the Tm can be determined is set out in the Examples. If polypeptides or proteins reach their Tm, the free energy change ΔG of protein folding or unfolding is equal to zero. At this point the polypeptide or protein molecules disappear into amorphous state and the protein chains cannot refold themselves. A general rule of thumb is that an increase in Tm is associated with increases in the free energy of maximal stability delta G(T*). Generally, proteins or polypeptides with a high Tm are more stable than those with lower Tm values. Increase in thermal stability leads to decreased denaturation of proteins and, thus, to, for example, improved storage conditions of said proteins. As shown in the Examples, thermal shift assays (TSA) can be used to measure the thermal stability of a protein and the increase in protein melting temperature (Tm) upon the binding of a stabilizing agent. In this assay, protein denaturation is monitored over a temperature gradient via an increase in fluorescence of SYPRO Orange, a dye which binds to hydrophobic residues that are exposed as the protein unfolds. A stabilizing compound binding to the protein leads to increased stability and an increase in Tm. The TSA as an exemplary method is disclosed herein below in the appended examples for determining the Tm in 20 mM HEPES buffer at pH 7.0. Preferably, the Tm is determined by TSA using differential scanning fluorimetry (DSF). In particular, the Tm is determined by TSA using DSF in HEPES buffer at pH 7.0 with a heating ramp rate of 0.5° C./min, wherein the protein has a concentration of 0.1 mg/ml. Protein stability was monitored over a temperature range from 30° C. to 70° C. The fluorescence of the fluorophore SYPRO orange (Bioworld, used at 5× concentration) was measured after 30 s equilibration at the new temperature. Delta Tm values of analyzed proteins can be calculated It is preferred that compositions that are compared to each other regarding their melting temperatures, comprise identical ingredients except the ingredient or analyte that is considered essential for the effect of increasing the melting temperature, such as ligands or any stabilizing agents, stabilizing additives, compounds or cofactors. For example, compositions that comprise an MHC protein without a peptide bound to said MHC protein have to be compared to an MHC protein without a peptide bound to said MHC protein plus R₁—COOH. It is further envisaged that compositions which comprise an MHC protein and a peptide, preferably a loading peptide bound to said MHC protein, have to be compared to an MHC protein with a peptide, preferably a loading peptide bound to said MHC protein and R₁—COOH.

In another embodiment of the first aspect of the present invention the composition is an aqueous solution consisting of or comprising the MHC protein, 20 mM HEPES buffer at pH 7, and an R₁—COOH or salt thereof, wherein the MHC protein comprised in the composition has an increased melting temperature (Tm), compared to a composition which is an aqueous solution consisting of the same MHC protein and 20 mM HEPES buffer at pH 7, wherein the melting temperature of the composition is increased by at least 0.5° C., 1.0° C., 1.5° C., 2.0° C., 2.5° C., 3.0° C., 3.5° C., 4.0° C., 4.5° C., 5.0° C., 5.5° C. or 6.0° C. Preferably, the R₁—COOH is comprised in the aqueous solution consisting of or comprising the MHC protein, 20 mM HEPES buffer at pH 7 in a concentration that it increases the melting temperature of the MHC protein comprised in the composition by at least 0.5° C., 1.0° C., 1.5° C., 2.0° C., 2.5° C., 3.0° C., 3.5° C., 4.0° C., 4.5° C., 5.0° C., 5.5° C. or 6.0° C.

In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the melting temperature of the composition is increased by at least 0.5° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the melting temperature of the composition is increased by at least 1.0° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the Tm of the composition is increased by at least 1.5° C. In one embodiment the composition is an aqueous solution consisting of or comprising the MHC protein, 20 mM HEPES buffer at pH 7, and the R₁—COOH or salt thereof, wherein the MHC comprised in the composition has an increased Tm, compared to a composition which is an aqueous solution consisting of or comprising the same MHC protein and 20 mM HEPES buffer at pH 7, wherein the Tm of the composition is increased by at least 2.0° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the Tm of the composition is increased by at least 2.5° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the melting temperature of the composition is increased by at least 3.0° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the Tm of the composition is increased by at least 3.5° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the Tm of the composition is increased by at least 4.0° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the Tm of the composition is increased by at least 4.5° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the Tm of the composition is increased by at least 5.0° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the Tm of the composition is increased by at least 5.5° C. In one embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC and 20 mM HEPES buffer at pH 7, wherein the Tm of the composition is increased by at least 6.0° C. In a preferred embodiment the composition is an aqueous solution consisting of or comprising the MHC protein, 20 mM HEPES buffer at pH 7, and the R₁—COOH or salt thereof, wherein the MHC protein comprised in the composition has an increased Tm, compared to a composition which is an aqueous solution consisting of or comprising the same MHC protein and 20 mM HEPES buffer at pH 7, wherein the Tm of the composition is increased by at least 4.5° C. or 5.5° C.

In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 0.5° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 1.0° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 1.5° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 2.0° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 2.5° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 3.0° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 3.5° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 4.0° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 4.5° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 5.0° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 5.5° C., compared to a composition which is an aqueous solution consisting of or comprising the same MHC protein and 20 mM HEPES buffer at pH 7. In one embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 6.0° C., compared to a composition which is an aqueous solution consisting of or comprising the same MHC protein and 20 mM HEPES buffer at pH 7. In a preferred embodiment the composition is an aqueous solution consisting of or comprising the MHC protein, 20 mM HEPES buffer at pH 7, and the R₁—COOH or salt thereof, wherein the R₁—COOH or salt thereof is comprised in the composition in an amount that the MHC protein comprised in the composition has a Tm that is increased by at least 4.5° C. or at least 5.5° C., compared to a composition which is an aqueous solution consisting of or comprising the same MHC protein and 20 mM HEPES buffer at pH 7.

Since the MHC proteins are particularly stable in the composition of the invention also in the absence of a peptide, the MHC protein of the first aspect of the invention is in one preferred embodiment not bound to a peptide. The MHC protein in the composition of the first aspect of the invention is properly folded and amenable to direct loading with a peptide, preferably with an MHC-I peptide or MHC-II peptide.

It was surprisingly found by the present inventors that the stabilizing effect of R₁—COOH or salt thereof combines with the stabilizing effect of loading a peptide into the MHC. Surprisingly it was observed that both effects are almost additive. Accordingly, the MHC protein of the composition of the first aspect may also be bound to a peptide. The peptide may be a loading peptide, and/or an MHC-I or MHC II peptide, which is bound to said MHC protein. Thus, in one embodiment the MHC protein of the composition of the first aspect of the invention is bound to a loading peptide which, for example, further stabilizes the MHC in the composition of the first aspect of the present invention. In a preferred embodiment the composition comprises an MHC protein and a loading peptide, which is a dipeptide. Even more preferably, the composition comprises an MHC protein and a loading peptide selected from the group consisting of GM, GY, GL or GF. In a most preferred embodiment the composition comprises an MHC protein and the loading peptide GM. In another embodiment the composition of the first invention comprises an MHC-I or MHC-II peptide. In another embodiment the composition comprises a loading peptide and an MHC-I or MHC II peptide.

In this embodiment of the first aspect of the present invention the composition consists of or comprises the MHC protein bound to a peptide, preferably bound to a loading peptide, and an Rr-COOH or salt thereof, wherein the MHC protein comprised in the composition has an increased melting temperature (Tm), compared to a composition comprising or consisting of the same MHC protein, wherein the melting temperature of the composition is increased by between 4° C. to 12° C., preferably at least 4.0° C., 4.5° C., 5.0° C., 5.5° C., 6.0° C., 6.5° C., 7.0° C., 7.5° C., 8.0° C., 8.5° C., 9.0° C., 9.5° C., 10.0° C., 10.5° C., 11.0° C., 11.5° C. or 12° C. Preferably, the R₁—COOH is comprised in or added to the composition consisting of or comprising the MHC protein bound to a peptide, preferably bound to a loading peptide, and an Rr-COOH or salt thereof, in an amount that it increases the melting temperature of the MHC protein comprised in the composition by at least 4.0° C., 4.5° C., 5.0° C., 5.5° C., 6.0° C., 6.5° C., 7.0° C., 7.5° C., 8.0° C., 8.5° C., 9.0° C., 9.5° C., 10.0° C., 10.5° C., 11.0° C., 11.5° C. or 12° C. In this context it is preferred that the R—COOH is a fatty acid, preferably a naturally occurring fatty acid. More preferably, the fatty acid comprises 3-9 carbon atoms. Even more preferably, the fatty acid comprises 3-8 or 4-8 carbon atoms. Most preferably, the fatty acid comprises 5-7 carbon atoms. In this context it is also preferred that the Rr-COOH is a C₂₋₂₁-alkyl-COOH selected from the group consisting of C₃-C₂₁-alkyl, preferably of propanoic acid, n-butanoic acid, iso-butanoic acid, propionic acid, butyric acid, valeric acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid or docosanoic acid. Preferably, the concentration of the Rr-COOH, preferably of the above mentioned Rr-COOH is about 5 mM to about 100 mM, about 10 mM to about 80 mM, about 15 mM to about 70 mM, about 20 mM to about 60 mM, about 25 mM to about 50 mM, about 30 mM to about 40 mM, or about 30 mM to about 35 mM. In a preferred embodiment the composition comprises an MHC protein and an Rr-COOH wherein the concentration of the Rr-COOH is about 5 mM to about 100 mM, about 10 mM to about 80 mM, about 15 mM to about 70 mM, about 20 mM to about 60 mM, or about 25 mM to about 50 mM. In a most preferred embodiment the composition comprises an MHC protein and an Rr-COOH wherein the concentration of the Rr-COOH is about 50 mM.

Preferably the fatty acid is at least one selected from the group consisting of butyric acid, valeric acid, heptanoic acid and hexanoic acid, preferably at a concentration of about 20 to 60 mM. Preferably the fatty acid is propionic acid, preferably at a concentration of about 80 to 150 mM. Preferably the fatty acid is octanoic acid, preferably at a concentration of about 5 to 60 mM.

In another embodiment the composition comprises an MHC protein and R₁—COOH, wherein the MHC protein is an MHC I or MHC II. In a preferred embodiment the MHC protein is a human leukocyte antigen (HLA). In another preferred embodiment the MHC protein is a multimer of MHC I, MHC II or HLA. It is preferred that the MHC I multimer is a dimer, a trimer or a tetramer. It is preferred that the MHC II multimer is a dimer, a trimer or a tetramer. It is even more preferred that the HLA multimer is a dimer, a trimer or a tetramer.

Stabilized MHC proteins that comprise artificial disulfide bridges have been described (WO 2020/053398 incorporated herein by reference in its entirety). It was surprisingly found by the inventors of the present invention that also the MHC proteins stabilized by an artificial disulfide bridge can be further stabilized if comprised in a composition of the first aspect of the present invention.

In another embodiment the composition comprises an MHC protein and R₁—COOH wherein the MHC is stabilized between one amino acid of the alpha1 domain and one amino acid of the alpha2 domain of said stabilized MHC protein in case of MHC I. In another embodiment the composition comprises an MHC and R₁—COOH wherein the MHC is stabilized between two amino acids of the alpha1 domain of said stabilized MHC protein in case of MHC I. In another embodiment the composition comprises an MHC protein and R₁—COOH, wherein the MHC protein is stabilized between one amino acid of the alpha1 domain and one amino acid of the alpha2 domain of said stabilized MHC protein in case of MHC I; and between two amino acids of the alpha1 domain of said stabilized MHC protein in case of MHC I. In another embodiment the composition comprises an MHC protein and R₁—COOH, wherein the MHC protein is stabilized between two amino acids of the alpha1 domain or the beta1 domain of said stabilized MHC protein in case of MHC II; and between one amino acid of the alpha1 domain and one amino acid of the beta1 domain of said stabilized MHC protein in case of MHC II.

In a preferred embodiment the composition comprises an MHC protein and an R₁—COOH, wherein the MHC protein is stabilized by one covalent bond between one amino acid of the alpha1 domain and one amino acid of the alpha2 domain of said stabilized MHC protein in case of MHC I. In another preferred embodiment the composition comprises an MHC protein and an R₁—COOH, wherein the MHC protein is stabilized by one covalent bond between two amino acids of the alpha1 domain of said stabilized MHC protein in case of MHC I. In another preferred embodiment the composition comprises an MHC protein and an R₁—COOH, wherein the MHC protein is stabilized by one covalent bond between one amino acid of the alpha1 domain and one amino acid of the alpha2 domain of said stabilized MHC protein in case of MHC I; and one covalent bond between two amino acids of the alpha1 domain of said stabilized MHC protein in case of MHC I. In another preferred embodiment the composition comprises an MHC protein and an R₁—COOH, wherein the MHC protein is stabilized by one covalent bond between two amino acids of the alpha1 domain or the beta1 domain of said stabilized MHC protein in case of MHC II; and one covalent bond between one amino acid of the alpha1 domain and one amino acid of the beta1 domain of said stabilized MHC protein in case of MHC II.

In another preferred embodiment the composition comprises an MHC protein and an R₁—COOH, wherein the MHC protein is stabilized by one covalent bond between α-helices. More preferably, the composition comprises an MHC protein and an R₁—COOH, wherein the MHC protein is stabilized by one covalent bond between a cysteine at IMGT position 84 and a cysteine at IMGT position 139 of MHC I; or by one covalent bond between a cysteine at IMGT position 51 and a cysteine at IMGT position 175. In another preferred embodiment the composition comprises an MHC protein and R₁—COOH, wherein the MHC protein is stabilized one covalent bond between α-helices and β-sheets of the alpha1 domain of MHC I. More preferably, the composition comprises an MHC protein and R₁—COOH, wherein the MHC protein is stabilized by one covalent bond between a cysteine at IMGT position 71 of MHC I and a cysteine at IMGT position 22 of MHC I. In another preferred embodiment the composition comprises an MHC protein and an R₁—COOH, wherein the MHC protein is stabilized by one covalent bond between α-helices and β-sheets. More preferably, the composition comprises an MHC protein and an R₁—COOH, wherein the MHC protein is stabilized by one covalent bond between a cysteine at IMGT position 71 of MHC I and a cysteine at IMGT position 22 of MHC I, and one covalent bond between a cysteine at IMGT position 51 of MHC I and a cysteine at IMGT position 175 of MHC I. More preferably, the composition comprises an MHC protein and an R₁—COOH, wherein the MHC protein is stabilized by one covalent bond between a cysteine at IMGT position 71 of MHC I and a cysteine at IMGT position 22 of MHC I, or wherein the MHC protein is stabilized by one covalent bond between a cysteine at IMGT position 84 and a cysteine at IMGT position 139 of MHC I. In another embodiment of the first aspect of the present invention the composition comprises an MHC protein and R₁—COOH, wherein the MHC protein is selected from group consisting of HLA-A, HLA-B HLA-C, HLA-E, HLA-F, HLA-G, HLA-J, HLA-K, and HLA-L. In a preferred embodiment the composition comprises a HLA-A and R₁—COOH wherein the HLA-A is selected from the group consisting of HLA-A1, HLA-A2, HLA-A3, and HLA-A11. In a preferred embodiment the composition comprises a HLA-A and R₁—COOH, wherein the HLA-A is selected from the group consisting of HLA-A*02; HLA-A*01 or HLA-A*03. In an even more preferred embodiment the composition comprises an HLA-A and an R₁—COOH, wherein the HLA-A is selected from the group consisting of HLA-A*02:01; HLA-A*01:01 or HLA-A*03:01. In a most preferred embodiment the composition comprises an HLA-A*02:01 and an R₁—COOH In another preferred embodiment the composition comprises a HLA-B and an R₁—COOH, wherein HLA-B is selected from the group consisting of HLA-B*07, HLA-B*08, HLA-B*15, HLA-B*35 and HLA-B*44. In amore preferred embodiment the composition comprises an HLA-B and R₁—COOH, wherein HLA-B is selected from the group consisting of HLA-B*07:02; HLA-B*08:01, HLA-B*15:01, HLA-B*35:01, HLA-B*44:03 and HLA-B*44:05.

In another embodiment of the first aspect of the present invention the composition comprises an MHC protein and R₁—COOH, wherein one or more MHC proteins are comprised in a complex. The term “complex” in context of the present invention refers to a higher order of MHC proteins, such as dimers, trimers, tetramers or multimers. In a preferred embodiment the composition comprises MHC protein tetramers and R₁—COOH. In a preferred embodiment the composition comprises MHC protein tetramers and R₁—COOH wherein the MHC proteins are bound to beads, filaments, nanoparticles, or other carriers.

In a preferred embodiment the composition comprises an HLA-A*02:01 and propionic acid or sodium salt thereof. In another preferred embodiment the composition comprises an HLA-A*02:01 and butyric acid or sodium salt thereof. In another preferred embodiment the composition comprises an HLA-A*02:01 and pentanoic acid or sodium salt thereof. In another preferred embodiment the composition comprises an HLA-A*02:01 and hexanoic acid or sodium salt thereof. In another preferred embodiment the composition comprises an HLA-A*02:01 and heptanoic acid or sodium salt thereof. In another preferred embodiment the composition comprises an HLA-A*02:01 and octanoic acid or sodium salt thereof. In another preferred embodiment the composition comprises an HLA-A*02:01 and nonanoic acid or sodium salt thereof. In another preferred embodiment the composition comprises an HLA-A*02:01 and 2-hexenoic acid or sodium salt thereof. In another preferred embodiment the composition comprises an HLA-A*02:01 and 3-hexenoic acid or sodium salt thereof. In another preferred embodiment the composition comprises an HLA-A*02:01 and 5-hexenoic acid or sodium salt thereof. In another preferred embodiment the composition comprises an HLA-A*02:01 and isopentanoic acid or sodium salt thereof. In another preferred embodiment the composition comprises an HLA-A*02:01 and 4-methylpentanoic acid or sodium salt thereof.

In a preferred embodiment the composition comprises an aqueous solution of HLA-A*02:01 and propionic acid or sodium salt thereof. In another preferred embodiment the composition comprises an aqueous solution of HLA-A*02:01 and butyric acid or sodium salt thereof. In another preferred embodiment the composition comprises an aqueous solution of HLA-A*02:01 and pentanoic acid or sodium salt thereof. In another preferred embodiment the composition comprises an aqueous solution of HLA-A*02:01 and hexanoic acid or sodium salt thereof. In another preferred embodiment the composition comprises an aqueous solution of HLA-A*02:01 and heptanoic acid or sodium salt thereof. In another preferred embodiment the composition comprises an aqueous solution of HLA-A*02:01 and octanoic acid or sodium salt thereof. In another preferred embodiment the composition comprises an aqueous solution of HLA-A*02:01 and nonanoic acid or sodium salt thereof. In another preferred embodiment the composition comprises an aqueous solution of HLA-A*02:01 and 2-hexenoic acid or sodium salt thereof. In another preferred embodiment the composition comprises an aqueous solution of HLA-A*02:01 and 3-hexenoic acid or sodium salt thereof. In another preferred embodiment the composition comprises an aqueous solution of HLA-A*02:01 and 5-hexenoic acid or sodium salt thereof. In another preferred embodiment the composition comprises an aqueous solution of HLA-A*02:01 and isopentanoic acid or sodium salt thereof. In another preferred embodiment the composition comprises an aqueous solution of HLA-A*02:01 and 4-methylpentanoic acid or sodium salt thereof.

In a preferred embodiment the composition comprises or consists of an aqueous solution of HLA-A*02:01 and propionic acid or sodium salt thereof and 20 mM HEPES buffer. In another preferred embodiment the composition comprises or consists of an aqueous solution of HLA-A*02:01 and butyric acid or sodium salt thereof and 20 mM HEPES buffer. In another preferred embodiment the composition comprises or consists of an aqueous solution of HLA-A*02:01 and pentanoic acid or sodium salt thereof and 20 mM HEPES buffer. In another preferred embodiment the composition comprises or consists of an aqueous solution of HLA-A*02:01 and hexanoic acid or sodium salt thereof and 20 mM HEPES buffer. In another preferred embodiment the composition comprises or consists of an aqueous solution of HLA-A*02:01 and heptanoic acid or sodium salt thereof and 20 mM HEPES buffer. In another preferred embodiment the composition comprises or consists of an aqueous solution of HLA-A*02:01 and octanoic acid or sodium salt thereof and 20 mM HEPES buffer. In another preferred embodiment the composition comprises or consists of an aqueous solution of HLA-A*02:01 and nonanoic acid or sodium salt thereof and 20 mM HEPES buffer. In another preferred embodiment the composition comprises or consists of an aqueous solution of HLA-A*02:01 and 2-hexenoic acid or sodium salt thereof and 20 mM HEPES buffer. In another preferred embodiment the composition comprises or consists of an aqueous solution of HLA-A*02:01 and 3-hexenoic acid or sodium salt thereof and 20 mM HEPES buffer. In another preferred embodiment the composition comprises or consists of an aqueous solution of HLA-A*02:01 and 5-hexenoic acid or sodium salt thereof and 20 mM HEPES buffer. In another preferred embodiment the composition comprises or consists of an aqueous solution of HLA-A*02:01 and isopentanoic acid or sodium salt thereof and 20 mM HEPES buffer. In another preferred embodiment the composition comprises or consists of an aqueous solution of HLA-A*02:01 and 4-methylpentanoic acid or sodium salt thereof and 20 mM HEPES buffer. As shown in the Examples, other buffer, such as MOPS or MES can be used in the present invention. In addition, the concentration of the buffer can vary, and the skilled person is able to adapt the concentration of the buffer in view of the screening methods as described in the examples.

In a preferred embodiment the composition comprises or consists of an aqueous solution of HLA-A*02:01 and propionic acid or sodium salt thereof and 20 mM HEPES buffer, wherein the solution has pH 7. In another preferred embodiment the composition comprises or consists of an aqueous solution of HLA-A*02:01 and butyric acid or sodium salt thereof and 20 mM HEPES buffer, wherein the solution has pH 7. In another preferred embodiment the composition comprises or consists of an aqueous solution of HLA-A*02:01 and pentanoic acid or sodium salt thereof and 20 mM HEPES buffer, wherein the solution has pH 7. In another preferred embodiment the composition comprises or consists of an aqueous solution of HLA-A*02:01 and hexanoic acid or sodium salt thereof and 20 mM HEPES buffer, wherein the solution has pH 7. In another preferred embodiment the composition comprises or consists of an aqueous solution of HLA-A*02:01 and heptanoic acid or sodium salt thereof and 20 mM HEPES buffer, wherein the solution has pH 7. In another preferred embodiment the composition comprises or consists of an aqueous solution of HLA-A*02:01 and octanoic acid or sodium salt thereof and 20 mM HEPES buffer, wherein the solution has pH 7. In another preferred embodiment the composition comprises or consists of an aqueous solution of HLA-A*02:01 and nonanoic acid or sodium salt thereof and 20 mM HEPES buffer, wherein the solution has pH 7. In another preferred embodiment the composition comprises or consists of an aqueous solution of HLA-A*02:01 and 2-hexenoic acid or sodium salt thereof and 20 mM HEPES buffer, wherein the solution has pH 7. In another preferred embodiment the composition comprises or consists of an aqueous solution of HLA-A*02:01 and 3-hexenoic acid or sodium salt thereof and 20 mM HEPES buffer, wherein the solution has pH 7. In another preferred embodiment the composition comprises or consists of an aqueous solution of HLA-A*02:01 and 5-hexenoic acid or sodium salt thereof and 20 mM HEPES buffer, wherein the solution has pH 7. In an even more preferred embodiment the composition comprises or consists of an aqueous solution of HLA-A*02:01 and isopentanoic acid or sodium salt thereof and 20 mM HEPES buffer, wherein the solution has pH 7. In another more preferred embodiment the composition comprises or consists of an aqueous solution of HLA-A*02:01 and 4-methylpentanoic acid or sodium salt thereof and 20 mM HEPES buffer, wherein the solution has pH 7.

In a preferred embodiment the composition comprises or consists of an aqueous solution of HLA-A*02:01 and 100 mM propionic acid or sodium salt thereof and 20 mM HEPES buffer, wherein the solution has pH 7. In another preferred embodiment the composition comprises or consists of an aqueous solution of HLA-A*02:01 and 50 mM butyric acid or sodium salt thereof and 20 mM HEPES buffer, wherein the solution has pH 7. In another preferred embodiment the composition comprises or consists of an aqueous solution of HLA-A*02:01 and 50 mM pentanoic acid or sodium salt thereof and 20 mM HEPES buffer, wherein the solution has pH 7. In another preferred embodiment the composition comprises or consists of an aqueous solution of HLA-A*02:01 and 50 mM hexanoic acid or sodium salt thereof and 20 mM HEPES buffer, wherein the solution has pH 7. In another preferred embodiment the composition comprises or consists of an aqueous solution of HLA-A*02:01 and 25 mM heptanoic acid or sodium salt thereof and 20 mM HEPES buffer, wherein the solution has pH 7. In another preferred embodiment the composition comprises or consists of an aqueous solution of HLA-A*02:01 and 10 mM octanoic acid or sodium salt thereof and 20 mM HEPES buffer, wherein the solution has pH 7. In another preferred embodiment the composition comprises or consists of an aqueous solution of HLA-A*02:01 and 5 mM nonanoic acid or sodium salt thereof and 20 mM HEPES buffer, wherein the solution has pH 7. In another preferred embodiment the composition comprises or consists of an aqueous solution of HLA-A*02:01 and 2-50 mM hexenoic acid or sodium salt thereof and 20 mM HEPES buffer, wherein the solution has pH 7. In another preferred embodiment the composition comprises or consists of an aqueous solution of HLA-A*02:01 and 50 mM 3-hexenoic acid or sodium salt thereof and 20 mM HEPES buffer, wherein the solution has pH 7. In another preferred embodiment the composition comprises or consists of an aqueous solution of HLA-A*02:01 and 50 mM 5-hexenoic acid or sodium salt thereof and 20 mM HEPES buffer, wherein the solution has pH 7. In an even more preferred embodiment the composition comprises or consists of an aqueous solution of HLA-A*02:01 and 50 mM isopentanoic acid or sodium salt thereof and 20 mM HEPES buffer, wherein the solution has pH 7. In another more preferred embodiment the composition comprises or consists of an aqueous solution of HLA-A*02:01 and 50 mM 4-methylpentanoic acid or sodium salt thereof and 20 mM HEPES buffer, wherein the solution has pH 7.

In the following in each case when the term “HLA-A*02:01_ds 84-139” is used this refers to a HLA comprising or consisting of amino acid sequence according to SEQ ID NO: 1.

In a preferred embodiment the composition comprises an HLA-A*02:01_ds 84-139 and propionic acid or sodium salt thereof. In another preferred embodiment the composition comprises an HLA-A*02:01_ds 84-139 and butyric acid or sodium salt thereof. In another preferred embodiment the composition comprises an HLA-A*02:01_ds 84-139 and pentanoic acid or sodium salt thereof. In another preferred embodiment the composition comprises an HLA-A*02:01_ds 84-139 and hexanoic acid or sodium salt thereof. In another preferred embodiment the composition comprises an HLA-A*02:01_ds 84-139 and heptanoic acid or sodium salt thereof. In another preferred embodiment the composition comprises an HLA-A*02:01_ds 84-139 and octanoic acid or sodium salt thereof. In another preferred embodiment the composition comprises an HLA-A*02:01_ds 84-139 and nonanoic acid or sodium salt thereof. In another preferred embodiment the composition comprises an HLA-A*02:01 ds 84-139 and 2-hexenoic acid or sodium salt thereof. In another preferred embodiment the composition comprises an HLA-A*02:01_ds 84-139 and 3-hexenoic acid or sodium salt thereof. In another preferred embodiment the composition comprises an HLA-A*02:01_ds 84-139 and 5-hexenoic acid or sodium salt thereof. In another preferred embodiment the composition comprises an HLA-A*02:01_ds 84-139 and isopentanoic acid or sodium salt thereof. In another preferred embodiment the composition comprises an HLA-A*02:01_ds 84-139 and 4-methylpentanoic acid or sodium salt thereof.

In a preferred embodiment the composition comprises an aqueous solution of HLA-A*02:01_ds 84-139 and propionic acid or sodium salt thereof. In another preferred embodiment the composition comprises an aqueous solution of HLA-A*02:01_ds 84-139 and butyric acid or sodium salt thereof. In another preferred embodiment the composition comprises an aqueous solution of HLA-A*02:01 ds 84-139 and pentanoic acid or sodium salt thereof. In another preferred embodiment the composition comprises an aqueous solution of HLA-A*02:01_ds 84-139 and hexanoic acid or sodium salt thereof. In another preferred embodiment the composition comprises an aqueous solution of HLA-A*02:01_ds 84-139 and heptanoic acid or sodium salt thereof. In another preferred embodiment the composition comprises an aqueous solution of HLA-A*02:01_ds 84-139 and octanoic acid or sodium salt thereof. In another preferred embodiment the composition comprises an aqueous solution of HLA-A*02:01_ds 84-139 and nonanoic acid or sodium salt thereof. In another preferred embodiment the composition comprises an aqueous solution of HLA-A*02:01_ds 84-139 and 2-hexenoic acid or sodium salt thereof. In another preferred embodiment the composition comprises an aqueous solution of HLA-A*02:01_ds 84-139 and 3-hexenoic acid or sodium salt thereof. In another preferred embodiment the composition comprises an aqueous solution of HLA-A*02:01_ds 84-139 and 5-hexenoic acid or sodium salt thereof. In another preferred embodiment the composition comprises an aqueous solution of HLA-A*02:01 and isopentanoic acid or sodium salt thereof. In another preferred embodiment the composition comprises an aqueous solution of HLA-A*02:01_ds 84-139 and 4-methylpentanoic acid or sodium salt thereof.

In a preferred embodiment the composition comprises or consists of an aqueous solution of HLA-A*02:01_ds 84-139 and propionic acid or sodium salt thereof and 20 mM HEPES buffer. In another preferred embodiment the composition comprises an aqueous solution of HLA-A*02:01_ds 84-139 and butyric acid or sodium salt thereof and 20 mM HEPES buffer. In another preferred embodiment the composition comprises or consists of an aqueous solution of HLA-A*02:01_ds 84-139 and pentanoic acid or sodium salt thereof and 20 mM HEPES buffer. In another preferred embodiment the composition comprises or consists of an aqueous solution of HLA-A*02:01_ds 84-139 and hexanoic acid or sodium salt thereof and 20 mM HEPES buffer. In another preferred embodiment the composition comprises or consists of an aqueous solution of HLA-A*02:01_ds 84-139 and heptanoic acid or sodium salt thereof and 20 mM HEPES buffer. In another preferred embodiment the composition comprises or consists of an aqueous solution of HLA-A*02:01 ds 84-139 and octanoic acid or sodium salt thereof and 20 mM HEPES buffer.

In another preferred embodiment the composition comprises or consists of an aqueous solution of HLA-A*02:01_ds 84-139 and nonanoic acid or sodium salt thereof and 20 mM HEPES buffer. In another preferred embodiment the composition comprises or consists of an aqueous solution of HLA-A*02:01_ds 84-139 and 2-hexenoic acid or sodium salt thereof and 20 mM HEPES buffer. In another preferred embodiment the composition comprises or consists of an aqueous solution HLA-A*02:01_ds 84-139 and 3-hexenoic acid or sodium salt thereof and 20 mM HEPES buffer. In another preferred embodiment the composition comprises or consists of an aqueous solution of HLA-A*02:01_ds 84-139 and 5-hexenoic acid or sodium salt thereof and 20 mM HEPES buffer. In another preferred embodiment the composition comprises or consists of an aqueous solution of HLA-A*02:01_ds 84-139 and isopentanoic acid or sodium salt thereof and 20 mM HEPES buffer. In another preferred embodiment the composition comprises an aqueous solution of HLA-A*02:01_ds 84-139 and 4-methylpentanoic acid or sodium salt thereof and 20 mM HEPES buffer.

In a preferred embodiment the composition comprises or consists of an aqueous solution of HLA-A*02:01_ds 84-139 and propionic acid or sodium salt thereof and 20 mM HEPES buffer, wherein the solution has pH 7. In another preferred embodiment the composition comprises or consists of an aqueous solution of HLA-A*02:01 ds 84-139 and butyric acid or sodium salt thereof and 20 mM HEPES buffer, wherein the solution has pH 7. In another preferred embodiment the composition comprises or consists of an aqueous solution of HLA-A*02:01_ds 84-139 and pentanoic acid or sodium salt thereof and 20 mM HEPES buffer, wherein the solution has pH 7. In another preferred embodiment the composition comprises or consists of an aqueous solution of HLA-A*02:01_ds 84-139 and hexanoic acid or sodium salt thereof and 20 mM HEPES buffer, wherein the solution has pH 7. In another preferred embodiment the composition comprises or consists of an aqueous solution of HLA-A*02:01_ds 84-139 and heptanoic acid or sodium salt thereof and 20 mM HEPES buffer, wherein the solution has pH 7. In another preferred embodiment the composition comprises or consists of an aqueous solution of HLA-A*02:01_ds 84-139 and octanoic acid or sodium salt thereof and 20 mM HEPES buffer, wherein the solution has pH 7. In another preferred embodiment the composition comprises or consists of an aqueous solution of HLA-A*02:01 ds 84-139 and nonanoic acid or sodium salt thereof and 20 mM HEPES buffer, wherein the solution has pH 7. In another preferred embodiment the composition comprises or consists of an aqueous solution of HLA-A*02:01_ds 84-139 and 2-hexenoic acid or sodium salt thereof and 20 mM HEPES buffer, wherein the solution has pH 7. In another preferred embodiment the composition comprises or consists of an aqueous solution of HLA-A*02:01_ds 84-139 and 3-hexenoic acid or sodium salt thereof and 20 mM HEPES buffer, wherein the solution has pH 7. In another preferred embodiment the composition comprises or consists of an aqueous solution of HLA-A*02:01 ds 84-139 and 5-hexenoic acid or sodium salt thereof and 20 mM HEPES buffer, wherein the solution has pH 7. In another preferred embodiment the composition comprises or consists of an aqueous solution of HLA-A*02:01_ds 84-139 and isopentanoic acid or sodium salt thereof and 20 mM HEPES buffer, wherein the solution has pH 7. In another preferred embodiment the composition comprises or consists of an aqueous solution of HLA-A*02:01_ds 84-139 and 4-methylpentanoic acid or sodium salt thereof and 20 mM HEPES buffer, wherein the solution has pH 7.

In a preferred embodiment the composition comprises or consists of an aqueous solution of HLA-A*02:01_ds 84-139 and propionic acid 100 mM or sodium salt thereof and 20 mM HEPES buffer, wherein the solution has pH 7. In another preferred embodiment the composition comprises or consists of an aqueous solution of HLA-A*02:01_ds 84-139 and 50 mM butyric acid or sodium salt thereof and 20 mM HEPES buffer, wherein the solution has pH 7. In another preferred embodiment the composition comprises or consists of an aqueous solution of HLA-A*02:01_ds 84-139 and 50 mM pentanoic acid or sodium salt thereof and 20 mM HEPES buffer, wherein the solution has pH 7. In another preferred embodiment the composition comprises or consists of an aqueous solution of HLA-A*02:01_ds 84-139 and 50 mM hexanoic acid or sodium salt thereof and 20 mM HEPES buffer, wherein the solution has pH 7. In another preferred embodiment the composition comprises or consists of an aqueous solution of HLA-A*02:01 ds 84-139 and 25 mM heptanoic acid or sodium salt thereof and 20 mM HEPES buffer, wherein the solution has pH 7. In another preferred embodiment the composition comprises or consists of an aqueous solution of HLA-A*02:01_ds 84-139 and 10 mM octanoic acid or sodium salt thereof and 20 mM HEPES buffer, wherein the solution has pH 7. In another preferred embodiment the composition comprises or consists of an aqueous solution of HLA-A*02:01 ds 84-139 and 5 mM nonanoic acid or sodium salt thereof and 20 mM HEPES buffer, wherein the solution has pH 7. In another preferred embodiment the composition comprises or consists of an aqueous solution of HLA-A*02:01_ds 84-139 and 2-50 mM hexenoic acid or sodium salt thereof and 20 mM HEPES buffer, wherein the solution has pH 7. In another preferred embodiment the composition comprises or consists of an aqueous solution of HLA-A*02:01_ds 84-139 and 50 mM 3-hexenoic acid or sodium salt thereof and 20 mM HEPES buffer, wherein the solution has pH 7. In another preferred embodiment the composition comprises or consists of an aqueous solution of HLA-A*02:01_ds 84-139 and 50 mM 5-hexenoic acid or sodium salt thereof and 20 mM HEPES buffer, wherein the solution has pH 7. In an even more preferred embodiment the composition comprises or consists of an aqueous solution of HLA-A*02:01_ds 84-139 and 50 mM isopentanoic acid or sodium salt thereof and 20 mM HEPES buffer, wherein the solution has pH 7. In another more preferred embodiment the composition comprises or consists of an aqueous solution of HLA-A*02:01_ds 84-139 and 50 mM 4-methylpentanoic acid or sodium salt thereof and 20 mM HEPES buffer, wherein the solution has pH 7.

In the following in each case when the term “HLA-A*02:01_ds 22-71” is used this refers to a HLA comprising or consisting of amino acid sequence according to SEQ ID NO: 2.

In a preferred embodiment the composition comprises HLA-A*02:01_ds 22-71 and propionic acid or sodium salt thereof. In another preferred embodiment the composition comprises HLA-A*02:01_ds 22-71 and butyric acid or sodium salt thereof. In another preferred embodiment the composition comprises a HLA-A*02:01_ds 22-71 and pentanoic acid or sodium salt thereof. In another preferred embodiment the composition comprises HLA-A*02:01_ds 22-71 and hexanoic acid or sodium salt thereof. In another preferred embodiment the composition comprises HLA-A*02:01_ds 22-71 and heptanoic acid or sodium salt thereof. In another preferred embodiment the composition comprises HLA-A*02:01_ds 22-71 and octanoic acid or sodium salt thereof. In another preferred embodiment the composition comprises a HLA-A*02:01_ds 22-71 and nonanoic acid or sodium salt thereof. In another preferred embodiment the composition comprises HLA-A*02:01_ds 22-71 and 2-hexenoic acid or sodium salt thereof. In another preferred embodiment the composition comprises HLA-A*02:01_ds 22-71 and 3-hexenoic acid or sodium salt thereof. In another preferred embodiment the composition comprises HLA-A*02:01_ds 22-71 and 5-hexenoic acid or sodium salt thereof. In another preferred embodiment the composition comprises HLA-A*02:01_ds 22-71 and isopentanoic acid or sodium salt thereof. In another preferred embodiment the composition comprises HLA-A*02:01_ds 22-71 and 4-methylpentanoic acid or sodium salt thereof.

In a preferred embodiment the composition comprises an aqueous solution of HLA-A*02:01_ds 22-71 and propionic acid or sodium salt thereof. In another preferred embodiment the composition comprises an aqueous solution of HLA-A*02:01_ds 22-71 and butyric acid or sodium salt thereof. In another preferred embodiment the composition comprises an aqueous solution of HLA-A*02:01_ds 22-71 and pentanoic acid or sodium salt thereof. In another preferred embodiment the composition comprises an aqueous solution of HLA-A*02:01_ds 22-71 and hexanoic acid or sodium salt thereof. In another preferred embodiment the composition comprises an aqueous solution of HLA-A*02:01_ds 22-71 and heptanoic acid or sodium salt thereof. In another preferred embodiment the composition comprises an aqueous solution HLA-A*02:01_ds 22-71 and octanoic acid or sodium salt thereof. In another preferred embodiment the composition comprises an aqueous solution of HLA-A*02:01_ds 22-71 and nonanoic acid or sodium salt thereof. In another preferred embodiment the composition comprises an aqueous solution of HLA-A*02:01 and 2-hexenoic acid or sodium salt thereof. In another preferred embodiment the composition comprises an aqueous solution of HLA-A*02:01_ds 22-71 and 3-hexenoic acid or sodium salt thereof. In another preferred embodiment the composition comprises an aqueous solution of HLA-A*02:01_ds 22-71 and 5-hexenoic acid or sodium salt thereof. In another preferred embodiment the composition comprises an aqueous solution of HLA-A*02:01_ds 22-71 and isopentanoic acid or sodium salt thereof. In another preferred embodiment the composition comprises an aqueous solution of HLA-A*02:01_ds 22-71 and 4-methylpentanoic acid or sodium salt thereof.

In a preferred embodiment the composition comprises or consists of an aqueous solution of HLA-A*02:01_ds 22-71 and propionic acid or sodium salt thereof and 20 mM HEPES buffer. In another preferred embodiment the composition comprises or consists of an aqueous solution of HLA-A*02:01_ds 22-71 and butyric acid or sodium salt thereof and 20 mM HEPES buffer. In another preferred embodiment the composition comprises or consists of an aqueous solution of HLA-A*02:01_ds 22-71 and pentanoic acid or sodium salt thereof and 20 mM HEPES buffer. In another preferred embodiment the composition comprises or consists of an aqueous solution of HLA-A*02:01_ds 22-71 and hexanoic acid or sodium salt thereof and 20 mM HEPES buffer. In another preferred embodiment the composition comprises or consists of an aqueous solution of HLA-A*02:01_ds 22-71 and heptanoic acid or sodium salt thereof and 20 mM HEPES buffer. In another preferred embodiment the composition comprises or consists of an aqueous solution of HLA-A*02:01 ds 22-71 and octanoic acid or sodium salt thereof and 20 mM HEPES buffer. In another preferred embodiment the composition comprises or consists of an aqueous solution of HLA-A*02:01_ds 22-71 and nonanoic acid or sodium salt thereof and 20 mM HEPES buffer. In another preferred embodiment the composition comprises or consists of an aqueous solution of HLA-A*02:01_ds 22-71 and 2-hexenoic acid or sodium salt thereof and 20 mM HEPES buffer. In another preferred embodiment the composition comprises or consists of an aqueous solution of HLA-A*02:01_ds 22-71 and 3-hexenoic acid or sodium salt thereof and 20 mM HEPES buffer. In another preferred embodiment the composition comprises or consists of an aqueous solution HLA-A*02:01_ds 22-71 and 5-hexenoic acid or sodium salt thereof and 20 mM HEPES buffer. In another preferred embodiment the composition comprises or consists of an aqueous solution of HLA-A*02:01_ds 22-71 and isopentanoic acid or sodium salt thereof and 20 mM HEPES buffer. In another preferred embodiment the composition comprises or consists of an aqueous solution of HLA-A*02:01_ds 22-71 and 4-methylpentanoic acid or sodium salt thereof and 20 mM HEPES buffer.

In a preferred embodiment the composition comprises or consists of an aqueous solution of HLA-A*02:01_ds 22-71 and propionic acid or sodium salt thereof and 20 mM HEPES buffer, wherein the solution has pH 7. In another preferred embodiment the composition comprises or consists of an aqueous solution of HLA-A*02:01_ds 22-71 and butyric acid or sodium salt thereof and 20 mM HEPES buffer, wherein the solution has pH 7. In another preferred embodiment the composition comprises or consists of an aqueous solution of HLA-A*02:01_ds 22-71 and pentanoic acid or sodium salt thereof and 20 mM HEPES buffer, wherein the solution has pH 7. In another preferred embodiment the composition comprises or consists of an aqueous solution of HLA-A*02:01_ds 22-71 and hexanoic acid or sodium salt thereof and 20 mM HEPES buffer, wherein the solution has pH 7. In another preferred embodiment the composition comprises or consists of an aqueous solution of HLA-A*02:01_ds 22-71 and heptanoic acid or sodium salt thereof and 20 mM HEPES buffer, wherein the solution has pH 7. In another preferred embodiment the composition comprises or consists of an aqueous solution of HLA-A*02:01_ds 22-71 and octanoic acid or sodium salt thereof and 20 mM HEPES buffer, wherein the solution has pH 7. In another preferred embodiment the composition comprises or consists of an aqueous solution of HLA-A*02:01_ds 22-71 and nonanoic acid or sodium salt thereof and 20 mM HEPES buffer, wherein the solution has pH 7. In another preferred embodiment the composition comprises or consists of an aqueous solution of HLA-A*02:01_ds 22-71 and 2-hexenoic acid or sodium salt thereof and 20 mM HEPES buffer, wherein the solution has pH 7. In another preferred embodiment the composition comprises or consists of an aqueous solution of HLA-A*02:01_ds 22-71 and 3-hexenoic acid or sodium salt thereof and 20 mM HEPES buffer, wherein the solution has pH 7. In another preferred embodiment the composition comprises or consists of an aqueous solution of HLA-A*02:01_ds 22-71 and 5-hexenoic acid or sodium salt thereof and 20 mM HEPES buffer, wherein the solution has pH 7. In another preferred embodiment the composition comprises or consists of an aqueous solution of HLA-A*02:01_ds 22-71 and isopentanoic acid or sodium salt thereof and 20 mM HEPES buffer, wherein the solution has pH 7. In another preferred embodiment the composition comprises or consists of an aqueous solution of HLA-A*02:01_ds 22-71 and 4-methylpentanoic acid or sodium salt thereof and 20 mM HEPES buffer, wherein the solution has pH 7.

In a preferred embodiment the composition comprises or consists of an aqueous solution of HLA-A*02:01_ds 22-71 and propionic acid 100 mM or sodium salt thereof and 20 mM HEPES buffer, wherein the solution has pH 7. In another preferred embodiment the composition comprises or consists of an aqueous solution of HLA-A*02:01 ds 22-71 and 50 mM butyric acid or sodium salt thereof and 20 mM HEPES buffer, wherein the solution has pH 7. In another preferred embodiment the composition comprises or consists of an aqueous solution of HLA-A*02:01_ds 22-71 and 50 mM pentanoic acid or sodium salt thereof and 20 mM HEPES buffer, wherein the solution has pH 7. In another preferred embodiment the composition comprises or consists of an aqueous solution of HLA-A*02:01_ds 22-71 and 50 mM hexanoic acid or sodium salt thereof and 20 mM HEPES buffer, wherein the solution has pH 7. In another preferred embodiment the composition comprises or consists of an aqueous solution of HLA-A*02:01_ds 22-71 and 25 mM heptanoic acid or sodium salt thereof and 20 mM HEPES buffer, wherein the solution has pH 7. In another preferred embodiment the composition comprises or consists of an aqueous solution of HLA-A*02:01_ds 22-71 and 10 mM octanoic acid or sodium salt thereof and 20 mM HEPES buffer, wherein the solution has pH 7. In another preferred embodiment the composition comprises or consists of an aqueous solution of HLA-A*02:01 ds 22-71 and 5 mM nonanoic acid or sodium salt thereof and 20 mM HEPES buffer, wherein the solution has pH 7. In another preferred embodiment the composition comprises or consists of an aqueous solution of HLA-A*02:01_ds 22-71 and 2-50 mM hexenoic acid or sodium salt thereof and 20 mM HEPES buffer, wherein the solution has pH 7. In another preferred embodiment the composition comprises or consists of an aqueous solution of HLA-A*02:01_ds 22-71 and 50 mM 3-hexenoic acid or sodium salt thereof and 20 mM HEPES buffer, wherein the solution has pH 7. In another preferred embodiment the composition comprises or consists of an aqueous solution of HLA-A*02:01_ds 22-71 and 50 mM 5-hexenoic acid or sodium salt thereof and 20 mM HEPES buffer, wherein the solution has pH 7. In an even more preferred embodiment the composition comprises or consists of an aqueous solution of HLA-A*02:01_ds 22-71 and 50 mM isopentanoic acid or sodium salt thereof and 20 mM HEPES buffer, wherein the solution has pH 7. In another more preferred embodiment the composition comprises or consists of an aqueous solution of HLA-A*02:01_ds 22-71 and 50 mM 4-methylpentanoic acid or sodium salt thereof and 20 mM HEPES buffer, wherein the solution has pH 7.

In a second aspect the invention further relates to the use of an R₁—COOH or salt thereof, for stabilizing an MHC protein, wherein R₁ is substituted or unsubstituted hydrocarbyl. Preferably, R₁ is unsubstituted and used as a stabilizing agent in the composition of the first aspect of the present invention. All embodiments specifically disclosed in context of the composition of the first aspect of the invention may be used for stabilizing an MHC protein according to the second aspect of the invention or may be combined with any embodiment specifically disclosed in context of the second aspect of the invention A third aspect of the invention relates to a method for stabilizing an MHC protein or a peptide binding fragment thereof. In certain embodiments, the method comprises the following steps:
(i) providing an MHC protein or a peptide binding fragment thereof;
(ii) contacting said MHC protein with an R₁—COOH or a salt thereof,
wherein R₁ is substituted or unsubstituted hydrocarbyl.

In preferred embodiments, the method comprises:
(i) providing a solution comprising an MHC protein or a peptide binding fragment thereof;
(ii) contacting said solution with said R₁—COOH or salt thereof,
   wherein R₁ is substituted or unsubstituted hydrocarbyl. In one embodiment the solution comprising an MHC protein or peptide binding fragment thereof is an aqueous solution.

The MHC protein or peptide binding fragment thereof can be folded or unfolded. Thus, the R₁—COOH or salt thereof may also be included facilitating the correct folding of the MHC protein or peptide binding fragment thereof in the solution, optionally a peptide may be included in the folding reaction, e.g. a loading peptide. Preferably, the MHC protein or peptide binding fragment thereof in the solution is folded and essentially peptide-free. In one embodiment the MHC protein or peptide binding fragment in the solution is peptide-free. All embodiments specifically disclosed in context of the composition of the first aspect of the invention may be used in the method for stabilizing an MHC protein or peptide binding fragment thereof according to the third aspect of the invention or may be combined with any embodiment specifically disclosed in context of the third aspect of the invention A fourth aspect of the invention relates to a kit comprising:
(i) an MHC protein;
(ii) an R₁—COOH or salt thereof; and wherein R₁ is substituted or unsubstituted hydrocarbyl; or
(iii) the composition of the first aspect of the invention.
   Preferably, R₁ is unsubstituted hydrocarbyl. The kit may further comprise one or more peptides. All embodiments specifically disclosed in context of the composition of the first aspect of the invention may be comprised in the kit according to the fourth aspect of the invention or may be combined with any embodiment specifically disclosed in context of the fourth aspect of the invention.
   In accordance with the above outlined embodiments the invention preferably relates to the following items
1. A composition comprising
   (i) a major histocompatibility complex (MHC) protein; and
   (ii) an R₁—COOH or salt thereof, preferably sodium salt; and
   wherein R₁ is substituted or unsubstituted hydrocarbyl.
2. The composition according to item 1, wherein the hydrocarbyl is C₂-C₂₁-hydrocarbyl, preferably linear C₂₋₂₁-alkyl, branched C₃₋₂₁-alkyl; linear C₂₋₂₁-alkenyl, branched C₃₋₂₁-alkenyl, linear C₂₋₂₁-alkynyl or branched C₄₋₂₁-alkynyl.
3. The composition according to claim item 2, wherein
   (i) the linear C₂₋₂₁-alkyl is linear C₂₋₈-alkyl, C₂—-alkyl, C₃₋₇-alkyl, or C₄₋₆-alkyl preferably ethyl, propyl, butyl, pentyl, hexyl, heptyl or octyl;
   (ii) the branched C₃₋₂₁-alkyl is branched C₃₋₈-alkyl, branched C₃₋₇-alkyl, branched C₄₋₆-alkyl, preferably iso-propyl, iso-butyl, sec-butyl, tert-butyl, 1-methyl-butyl, 2-methyl-butyl, 3-methyl-butyl, 1,1-dimethyl-propyl, 2,2-dimethyl-propyl, 1-ethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2, trimethyl-propyl, 1-ethyl-2-methyl-propyl, 1-methyl-hexyl, 2-methyl-hexyl, 3-methyl-hexyl, 4-methyl-hexyl, 5-methyl-hexyl, 1,1-dimethyl-pentyl, 1,2-dimethyl-pentyl, 1,3-dimethyl-pentyl, 1,4-dimethyl-pentyl, 2,2-dimethyl-pentyl, 2,3-dimethyl-pentyl, 2,4-dimethyl-pentyl, 3,3-dimethyl-pentyl, 3,4-dimethyl-pentyl, 1-ethyl-pentyl, 2-ethyl-pentyl, 3-ethyl-pentyl, 1,1,2-trimethyl-butyl, 1,1,3-trimethyl-butyl, 1,2,3-trimethyl-butyl, 1,2,2-trimethyl-butyl, 2,2,3-trimethyl-butyl, 1-methyl-1-ethyl-butyl, 1-ethyl-2-methyl-butyl, 1-ethyl-3-methyl-butyl, 1-methyl-2-ethyl-butyl, 2-methyl-2-ethyl-butyl, 1-propy-butyl, 1-methyl-heptyl, 2-methyl-heptyl, 3-methyl-heptyl, 4-methyl-heptyl, 5-methyl-heptyl, 6-methyl-heptyl, 1,1-dimethyl-hexyl, 1,2-dimethyl-hexyl, 1,3-dimethyl-hexyl, 1,4-dimethyl-hexyl, 1,5-dimethyl-hexyl, 2,2-dimethyl-hexyl, 2,3-dimethyl-hexyl, 2,4-dimethyl-hexyl, 2,5-dimethyl-hexyl, 3,3-dimethyl-hexyl, 3,4-dimethyl-hexyl, 3,5-dimethyl-hexyl, 4,4-dimethyl-hexyl, 4,5-dimethyl-hexyl, 5,5-dimethyl-hexyl, 1-ethyl-hexyl, 2-ethyl-hexyl, 3-ethyl-hexyl or 4-ethyl-hexyl;
   (iii) the linear C₂₋₂₁-alkenyl is linear C₂₋₈-alkenyl, linear C₂₋₇-alkenyl, linear C₃₋₇-alkenyl, linear C₄₋₆-alkenyl, preferably ethenyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 1-heptenyl, 4-heptenyl, 5-heptenyl, 5-heptenyl, 6-heptenyl, 1-octenyl, 2-octenyl, 3-octenyl, 4-octenyl, 5-octenyl, 6-octenyl or 7-octenyl;
   (iv) the branched C₃₋₂₁-alkenyl is branched C₃₋₈-alkenyl, branched C₃₋₇-alkenyl, branched C₄₋₆-alkenyl, preferably-methylethenyl, 1-methyl-1-propenyl, 2-methyl-1-propenyl, 1-methyl-2-propenyl, 2-methyl-2-propenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-Dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, or 1-ethyl-2-methyl-2-propenyl,
   (v) the linear C₂₋₂₁-alkynyl is linear C₂₋₈-alkynyl, linear C₂₋₇-alkynyl, linear C₃₋₇-alkynyl, linear C₄-6-alkynyl, preferably ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 2-heptynyl, 1-heptynyl, 4-heptynyl, 5-heptynyl, 5-heptynyl, 6-heptynyl, 1-octynyl, 2-octynyl, 3-octynyl, 4-octenyl, 5-octynyl, 6-octynyl or 7-octynyl; or
   (vi) the branched C₄₋₂₁-alkynyl is branched C₄-s-alkynyl, branched C₃-s-alkynyl, branched C₄-6-alkynyl, preferably 1-methyl-2-propynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl 1-ethyl-3-butynyl, 2-ethyl-3-butynyl or 1-ethyl-1-methyl-2-propynyl.
4. The composition according to any of items 1-3, wherein:
   (i) in the linear C₂₋₂₁-alkenyl or in the branched C₃₋₂₁-alkenyl, a double bond is between the C₁ and C₂ atom or between the C₂ and C₃ atom of the hydrocarbyl; and/or
   (ii) in the linear C₂₋₂₁-alkynyl or in the branched C₄₋₂₁-alkynyl, a triple bond is between the C₁ and C₂ atom or between the C₂ and C₃ atom of the hydrocarbyl; and/or
   (iii) the hydrocarbyl is substituted with one or more substituents selected from the group consisting of halogen, in particular F, Cl, or Br; —OH; —O—R₄; ═O; —NO₂; —NR₂R₃, wherein R₂ and R₃ are independently selected from H, and hydrocarbyl; and —N—HR₄, wherein R₄ is selected from H and hydrocarbyl.
5. The composition according to any one of items 1-4, wherein the C₂₋₂₁-alkyl-COOH is selected from the group consisting of propanoic acid, n-butanoic acid, iso-butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, preferably propanoic acid, n-butanoic acid, pentanoic acid, hexanoic acid, heptanoic acid, octanoic acid or nonanoic acid.
6. The composition according to any one of items 1-5, wherein the
   (i) branched C₃₋₂₁-alkyl-COOH is selected from the group consisting of isobutanoic acid, isopentanoic acid, 3-methyl-pentanoic acid or 4-methyl-pentanoic acid;
   (ii) C₂₋₂₁-alkenyl-COOH is selected from the group consisting of 2-hexenoic acid, 3-hexenoic acid and 5-hexenoic acid, preferably 2-hexenoic acid or 3-hexenoic acid.
7. The composition according to any one of items 1-6, wherein the concentration of an R₁—COOH is about 5 mM to about 100 mM, about 10 mM to about 80 mM, about 15 mM to about 70 mM, about 20 mM to about 60 mM, about 25 mM to about 50 mM, about 30 mM to about 40 mM, about 30 mM to about 35 mM, preferably about 50 mM.
8. The composition according to any one of items 1-7, wherein the composition is an aqueous solution.
9. The composition according to any one of items 1-8, wherein the pH of the composition is in the range of pH 6 to pH 8, pH 6 to pH 7.5, or pH 6.4 to pH 7.5, preferably pH 6.4 to pH 7.5, more preferably pH 6 to 7.2, or most preferably pH 6.4 to pH 7.2.
10. The composition according to any one of items 8 or 9, comprising a buffer substance selected from the group consisting of ACES, ADA, BES, Bis-Tris Propane, DIPSO, EPPS, HEPPSO, Imidazol, MOBS, MES, Bis-Tris, MOPS, TRIS, MOPSO, Phosphate, PIPES, POPSO, TAPSO, TEA, TES, Tricine or HEPES buffer, preferably MES, Bis-Tris; MOPS, HEPES or TRIS.
11. The composition according to any one of items 8-10, wherein the R₁—COOH or salt thereof is comprised in the composition in a concentration that it
   (i) stabilizes the MHC protein comprised in the composition; and/or
   (ii) increases the melting temperature of the MHC protein comprised in the composition by at least 0.5° C., 1.0° C., 1.5° C., 2.0° C., 2.5° C., 3.0° C., 3.5° C., 4.0° C., 4.5° C., 5.0° C., 5.5° C. or 6.0° C.
12. The composition according to any of items 8-10, wherein the MHC comprised in the composition has an increased melting temperature compared to an MHC comprised in the same composition lacking the R₁—COOH or salt thereof, preferably the melting temperature of the composition is increased by at least 0.5° C., 1.0° C., 1.5° C., 2.0° C., 2.5° C., 3.0° C., 3.5° C., 4.0° C., 4.5° C., 5.0° C., 5.5° C. or 6.0° C.
13. The composition according to any of item 8-10, wherein the MHC protein comprised in the composition has an increased melting temperature (Tm), wherein the Tm of said MHC protein is increased, if the Tm of the MHC protein in a composition according to (a) is higher than the Tm of the MHC protein in a composition according to (b):
   (a) an aqueous composition consisting of the MHC protein, 20 mM HEPES buffer at pH 7, and R₁—COOH or salt thereof,
   (b) an aqueous composition consisting of the same MHC protein as in (a) and 20 mM HEPES buffer at pH 7; and
   preferably the melting temperature of the composition is increased by at least 0.5° C., 1.0° C., 1.5° C., 2.0° C., 2.5° C., 3.0° C., 3.5° C., 4.0° C., 4.5° C., 5.0° C., 5.5° C. or 6.0° C.
14. The composition according to any of items 1-13, wherein the MHC protein is an MHC I or MHC II molecule, preferably a human leukocyte antigen (HLA) or a multimer of MHC I, MHC II or HLA, such as a dimer, a trimer or a tetramer.
15. The composition according to any of items 1-14, wherein the MHC protein is stabilized, preferably by one or more covalent bonds:
   (i) between one amino acid of the alpha1 domain and one amino acid of the alpha2 domain of said stabilized MHC protein in case of MHC I; and/or
   (ii) between two amino acids of the alpha1 domain of said stabilized MHC protein in case of MHC I; or
   (iii) between two amino acids of the alpha1 domain or the beta1 domain of said stabilized MHC protein in case of MHC II; and/or
   (iv) between one amino acid of the alpha1 domain and one amino acid of the beta1 domain of said stabilized MHC protein in case of MHC II.
16. The composition according to item 15, wherein said covalent bond is:
   (i) between α-helices, preferably between a cysteine at IMGT position 84 and a cysteine at IMGT position 139 of MHC I;
   (ii) between α-helices and β-sheets of the alpha1 domain of MHC I, preferably between a cysteine at IMGT position 71 of MHC I and a cysteine at IMGT position 22 of MHC I;
   (iii) between α-helices, preferably by mutating an amino acid at position 51 of MHC I and an amino acid at position 175 of MHC I into cysteines; or
   (iv) between α-helices and β-sheets, preferably between a cysteine at IMGT position 71 of MHC I and a cysteine at IMGT position 22 of MHC I, and between α-helices, preferably between a cysteine at IMGT position 51 of MHC I and a cysteine at IMGT position 175 of MHC I.
17. The composition according to any one of items 1-16, wherein the MHC protein is selected from group consisting of HLA-A, HLA-B HLA-C; HLA-E; HLA-F; HLA-G; HLA-J; HLA-K, and HLA-L, wherein preferably HLA-A is selected from the group consisting of HLA-A1, HLA-A2, HLA-A3, and HLA-A11, more preferably HLA-A*02, even more preferably HLA-A*02:01; HLA-A*01:01 or HLA-A*03:01; and wherein preferably HLA-B is selected from the group consisting of HLA-B*07, HLA-B*08, HLA-B*15, HLA-B*35 and HLA-B*44, more preferably HLA-B*07:02; HLA-B*08:01, HLA-B*15:01, HLA-B*35:01 and HLA-B*44:05
18. The composition according to any one of items 1-17, wherein one or more MHC proteins are comprised in a complex, preferably the MHC proteins are bound to beads, filaments, nanoparticles, or other carriers.
19. The composition according to any one of items 1-18, wherein the composition further comprises at least one peptide, preferably a loading peptide selected from the group consisting of glycine-methionine (GM), glycine-tyrosine (GY), glycine-leucine (GL) and glycine-phenylalanine (GF), more preferably the loading peptide is GM.
20. The composition according to anyone of items 1-19, wherein the MHC protein comprises or consists of SEQ ID NO: 1 and SEQ ID NO: 2.
21. Use of an R₁—COOH or salt thereof, for stabilizing an MHC protein, wherein R₁ is substituted or unsubstituted hydrocarbyl.
22. Use according to item 21, wherein R₁ is C₂-C₂₁-hydrocarbyl.
23. A method for stabilizing an MHC protein, preferably an MHC I or MHC II, more preferably a HLA, comprising the following steps:
   (i) providing a solution comprising said MHC protein;
   (ii) contacting said solution with an R₁—COOH or salt thereof,
   wherein R₁ is substituted or unsubstituted hydrocarbyl.
24. The method according to item 23, wherein R₁ is C₂-C₂₁-hydrocarbyl.
25. The method according to item 23 or 24, further comprising the step of loading the MHC protein with a peptide, preferably an MHC-I peptide or MHC-II peptide.
26. A kit comprising:
   (i) an MHC protein; and
   (ii) an R₁—COOH or salt thereof, preferably sodium salt; or
   (iii) the composition of the first aspect of the invention; and
   (iv) optionally a peptide, preferably a loading peptide, an MHC I peptide and/or an MHC II peptide,
   wherein R₁ is substituted or unsubstituted hydrocarbyl.
27. The kit according to item 26, wherein R₁ is C₂-C₂₁-hydrocarbyl.

Throughout the instant application, the term “and/or” is a grammatical conjunction that is to be interpreted as encompassing that one or more of the cases it connects may occur. For example, the wording “such native sequence proteins can be prepared using standard recombinant and/or synthetic methods” indicates that native sequence proteins can be prepared using standard recombinant and synthetic methods or native sequence proteins can be prepared using standard recombinant methods or native sequence proteins can be prepared using synthetic methods.

Furthermore, throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Furthermore the indefinite article “a” or “an” does not exclude a plurality. The mere fact that certain measures are recited in mutually different dependent claims does not indicate that a combination of these measures cannot be used to advantage

The following examples are merely illustrative of the present invention and should not be construed to limit the scope of the invention as indicated by the appended claims in any way.

### EXAMPLES

### Example 1

All following experiments were performed with recombinant, disulfide bridge stabilized HLA-A*02:01, namely HLA-A*02:01_ds 22-71 in which a disulfide bond between positions 22 and 71 was introduced. HLA molecule HLA-A*02:01_ds 84-139 in which a disulfide bond between positions 84 and 139 was introduced was also tested. Proteins were refolded according to a published protocol (Garboczi et al.; “HLA-A2-peptide complexes: Refolding and crystallization of molecules expressed in Escherichia coli and complexed with single antigenic peptides”; PNAS, vol. 89, 3429-3433, 1992) in a buffer containing 100 mM HEPES, 400 mM arginine, 2 mM EDTA, 5 mM reduced and 0.5 mM oxidized glutathione. For HLA-A*02:01 ds 84-139, 10 mM GM dipeptide was included in the refolding reaction as described by Moritz et al. 2019. After refolding the protein was concentrated by ultrafiltration and purified by size exclusion chromatography. Fatty acids were dissolved in or diluted with water and neutralized with NaOH to give stock solution of 0.5 M pH 7.

### Screening for HLA-A*02:01 Stabilizing Compounds.

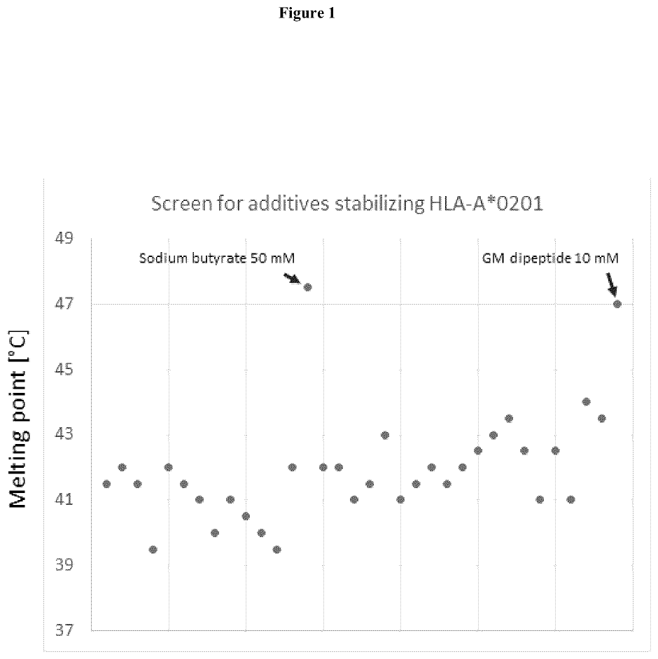

In order to find optimal storage and handling conditions for peptide free recombinant HLA-A*02:01 preparations, different compounds were screened in a thermal shift assay (TSA). Measurements were performed in the Applied Biosystems 7500 real time PCR instrument at a protein concentration of 0.1 mg/ml in a buffer of 20 mM Tris pH 8.0, 150 mM NaCl. The additives indicated in table 3 were added or alternatively water was added as a control. The raw data was exported and the analysis of the melting temperature was performed in Microsoft Excel. Protein stability was monitored over a temperature range from 30° C. to 70° C. The temperature was increased in 0.5° C. steps and the fluorescence of the fluorophore SYPRO orange (bio-world, Cat. No. 41900192-1, used at 5× concentration) was measured after 30 s equilibration at the new temperature. The temperature at which the maximal increase of fluorescence compared to the previous measurement was observed, is defined as melting point in the respective composition. Only two of the 33 compounds listed in Table 3 lead to a marked increase in protein stability (see FIG. 1): (i) the dipeptide glycine-methionine, which was previously described to stabilize HLA-A*0201 ds 84-139; and (ii) sodium butyrate, which even showed an increased stabilizing effect compared to the dipeptide glycine-methionine. The increased stabilization of the HLA molecule by the fatty acid was surprising since the increased stabilization was demonstrated in comparison to well-known protein stabilizers, such as glycerol, arginine or trehalose.

TABLE 3
Additives tested for HLA-A*0201 stabilization in thermal shift assay

| | Final | | Final |
|---|---|---|---|
| Additive | concentration | Additive | concentration |
| H2O | | Glycine | 0.1M |
| Calcium chloride | 10 mM | Histidine | 10 mM |
| Magnesium chloride | 10 mM | Imidazol | 20 mM |
| Ammonium chloride | 0.1M | Cysteamin | 10 mM |
| Ammonium acetate | 0.1M | Urea | 20 mM |
| Potassium chloride | 0.1M | Guanidinium | 20 mM |
| Lithium chloride | 0.1M | hydrochloride | |
| Sodium chloride | 0.2M | EDTA | 10 mM |
| Sodium phosphate | 0.1M | Trehalose | 3% (w/v) |
| Sodium sulfate | 0.1M | Sucrose | 3% (w/v) |
| Potassium phosphate | 50 mM | Dextrose | 3% (w/v) |
| Sodium citrate | 50 mM | Galactose | 1.5% (w/v) |
| Sodium acetate | 50 mM | Sorbitol | 3% (w/v) |
| Sodium butyrate | 50 mM | Ethanol | 5% (w/v) |
| Asparagine | 10 mM | Isopropanol | 5% (w/v) |
| Proline | 10 mM | Glycerol | 5% (w/v) |
| Arginine | 40 mM | PEG4000 | 3% (w/v) |
| | | GM Dipeptide | 10 mM |

### Example 2: Comparison of the Stabilizing Effect of Different Fatty Acids

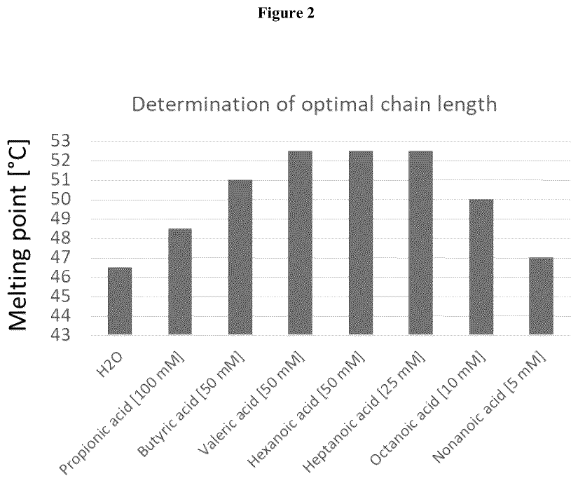

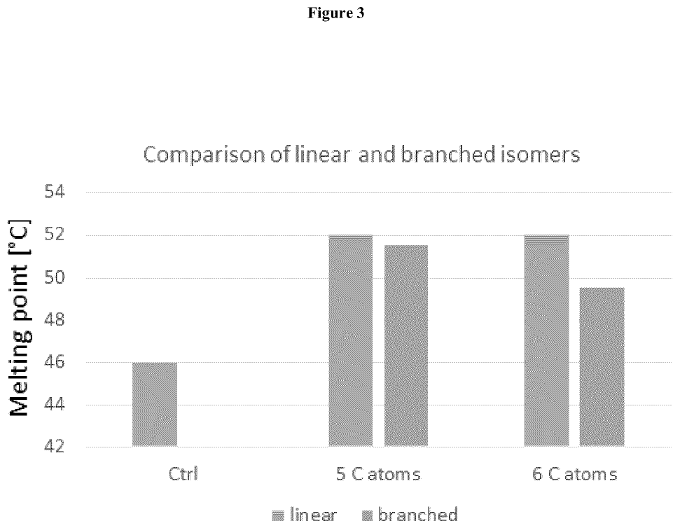

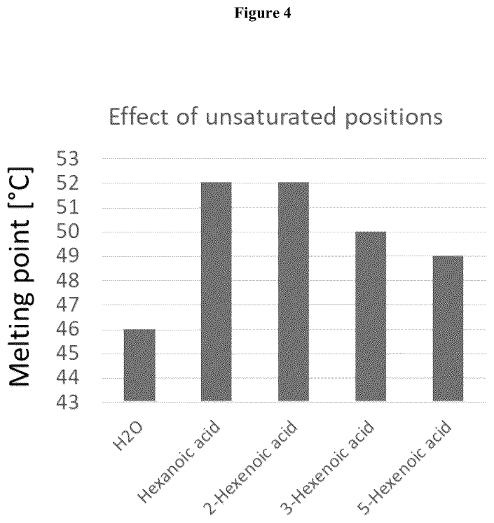

To test if other fatty acids also stabilize the HLA molecules, fatty acids with different chain lengths were screened in the TSA described above. In this experiment, the final gel filtration of the protein purification protocol was done in 20 mM HEPES pH 7.0 and the protein was used in this buffer at a concentration of 0.1 mg/ml. Fatty acids were added in a concentration range of 5-100 mM or water was added as control. The melting points observed at the optimal concentration for each additive are shown in FIG. 2. All fatty acids tested stabilize the HLA molecule. The fatty acids with 3-8 carbon atoms in length have strong stabilizing properties. The stabilizing effect is further increased by the fatty acids with 5-7 carbon atoms in length. With increasing chain length, the optimal concentration of the fatty acid moves towards lower concentration levels of the fatty acid. In order to show that the stabilization also applies for branched fatty acids or unsaturated fatty acids, the stabilization of the linear, saturated fatty acids was compared to isomers with branched chains or to unsaturated variants with the same chain length. The fatty acids with branched chains (isovaleric acid or 4-methyl-valeric acid) showed a stabilizing effect on the HLA molecule (FIG. 3). Therefore, the stabilization was also observed for branched fatty acids although the measured melting points indicate that linear chains tested show stronger stabilization of the HLA molecules than their branched isomers (FIG. 3). The unsaturated variants tested also showed a stabilizing effect on the HLA molecule. The closer the double bond (C═C) was situated to the carboxyl group, the stronger the stabilizing effect was (FIG. 4). The unsaturated fatty acids of the hexanoic acid thus, also showed a stabilizing effect on the HLA molecule. Accordingly, the linear, branched, saturated and unsaturated fatty acids stabilized the HLA molecule.

### Example 3: Stabilization is Observed for Different Ds-HLA Constructs

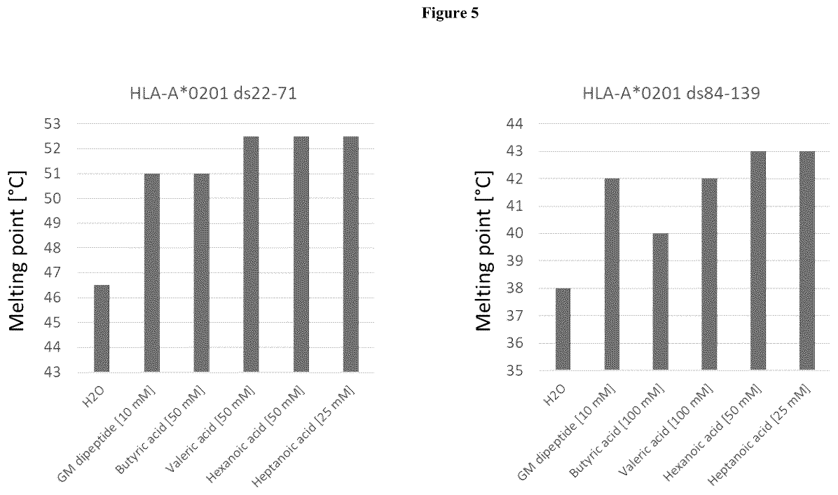

The stabilizing effect of fatty acids was also tested on a further HLA molecule, i.e. HLA-A*02:01_ds 84-139 in which a disulfide bond between positions 84 and 139 was introduced. Whereas the mutant with a disulfide bridge between the positions 22 and 71 can be refolded without addition of any peptide, the version containing a disulfide bond between the positions 84 and 139 required a dipeptide for refolding. The dipeptide was removed by gel filtration in 20 mM HEPES pH 7.0 prior to TSA measurements. TSA experiments as described in Example 2 were used to test the stabilizing effect of the fatty acids on HLA-A*02:01_ds 22-71 compared to HLA-A*02:01_ds 84-139 (FIG. 5). The effect of very short chained compounds (4 and 5 carbons) was increased in HLA-A*02:01_ds 22-71 compared to HLA-A*02:01_ds 84-139. Nonetheless, the melting point of both proteins was increased by 5-6° C. compared to the control conditions. Thus, the fatty acids stabilized both of the HLA molecules.

### Example 4: Fatty acids can further stabilize MHC molecules bound to a loading peptide

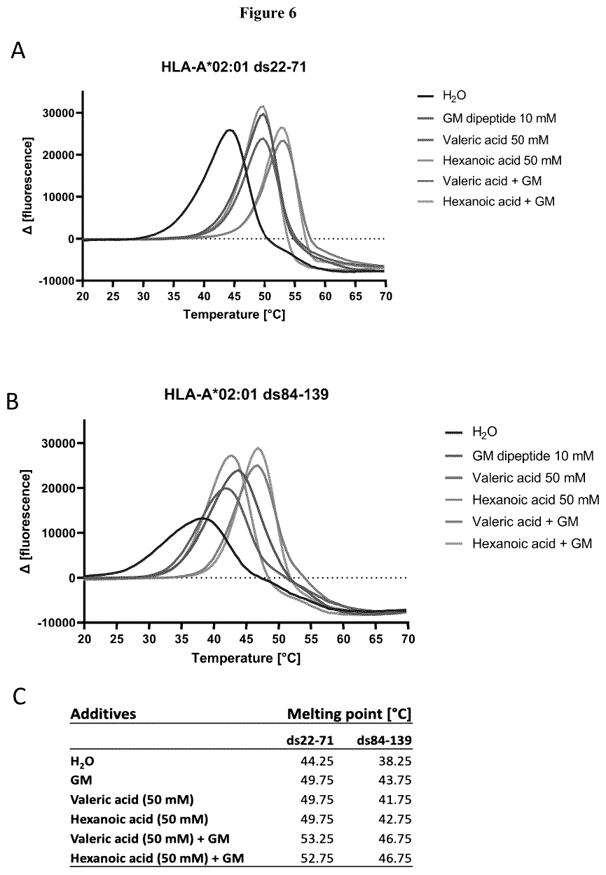

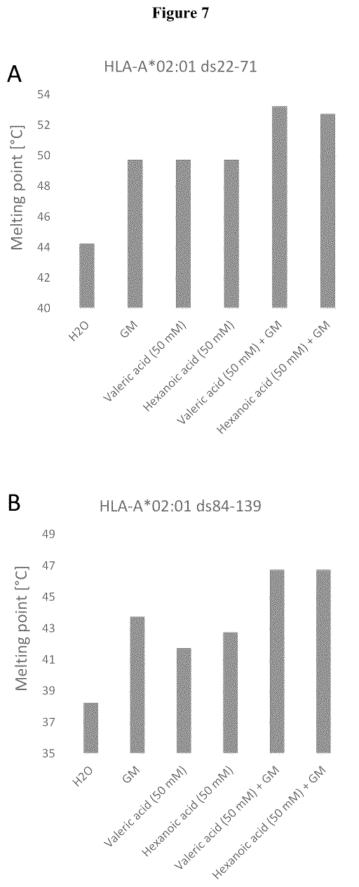

In order to test whether a combination of loading peptide and fatty acid can be used to stabilize MHC proteins for subsequent loading with an antigenic peptide, such as MHC-I or MCH-II peptides as defined above, TSA was performed as described in Example 1. HLA-A*02:01_ds_22-71_and HLA-A*02:01 ds_84-139, respectively were analyzed with and without the addition of a fatty acid, e.g. sodium butyrate, valeric acid or hexanoic acid (50 mM final concentration), and a dipeptide (GM, 10 mM final concentration). Stabilization of the HLA proteins was observed with both additives. In samples containing both additives an even stronger stabilization was observed, demonstrating that fatty acids can stabilize HLA molecules bound to loading peptides. Reference is made to FIGS. 5 to 7.

### Example 5: Stabilization in Buffers with Different pH

In a parallel TSA-screen with 40 different buffer conditions (MES pH between 5.8 to 7.2; BisTris pH between 5.8 to 7.2; MOPS pH between 6.5 to 7.9; HEPES pH 6.8 to 8.2; and Tris pH between 7.6 to 9.0), a clear link between pH conditions and stability of recombinant HLA was detected. In this experiment, a solution of 0.1 mg/ml HLA-A*0201 ds 22-71 in 20 mM HEPES pH 7.0 was supplemented with 0.1 volume of 1 M stock solution of the respective buffer.

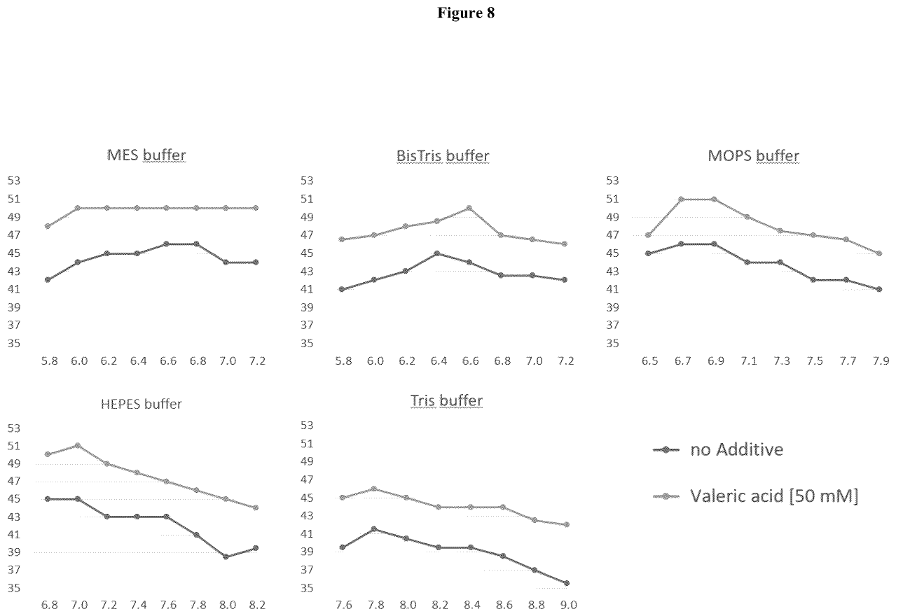

Over all buffer systems tested, pH values between 6.4 and 7.0 resulted in the highest protein stability with a progressive decline towards more basic or acidic conditions (FIG. 8). This pH range was also found to be optimal for peptide loaded HLA molecules. In order to test whether HLA is stabilized in different pH conditions by the fatty acids, a TSA-screen with the same 40 different buffer conditions was performed as described above. The stabilizing effect obtained by valeric acid (50 mM) was increased in all pH conditions tested compared to tests without the fatty acid (FIG. 8). The stabilizing effect in the pH range of 6 to 7.2 was particularly increased. Accordingly, the stabilizing effect of the fatty acid on the HLA molecule was observed under different pH conditions.

1. A composition comprising
(i) a major histocompatibility complex (MHC) protein; and
(ii) an R₁—COOH or salt thereof; and
wherein R₁ is substituted or unsubstituted hydrocarbyl.

2. The composition according to claim 1, wherein the hydrocarbyl is C₂-C₂₁-hydrocarbyl, optionally linear C₂₋₂₁-alkyl, branched C₃₋₂₁-alkyl; linear C₂₋₂₁-alkenyl, branched C₃₋₂₁-alkenyl, linear C₂₋₂₁-alkynyl or branched C₄₋₂₁-alkynyl.

3. The composition according to claim 2, wherein
(i) the linear C₂₋₂₁-alkyl is linear C₂₋₈-alkyl;
(ii) the branched C₃₋₂₁-alkyl is branched C₃₋₈-alkyl;
(iii) the linear C₂₋₂₁-alkenyl is linear C₂₋₈-alkenyl;
(iv) the branched C₃₋₂₁-alkenyl is branched C₃₋₈-alkenyl;
(v) the linear C₂₋₂₁-alkynyl is linear C₂₋₈-alkynyl; or
(vi) the branched C₄₋₂₁-alkynyl is branched C₄-8-alkynyl.

4. The composition according to claim 1, wherein:
(i) in the linear C₂₋₂₁-alkenyl or in the branched C₃₋₂₁-alkenyl, a double bond is between the C₁ and C₂ atom or between the C₂ and C₃ atom of the hydrocarbyl; and/or
(ii) in the linear C₂₋₂₁-alkynyl or in the branched C₄₋₂₁-alkynyl, a triple bond is between the C₁ and C₂ atom or between the C₂ and C₃ atom of the hydrocarbyl; and/or
(iii) the hydrocarbyl is substituted with one or more substituents selected from the group consisting of halogen, in particular F, Cl, or Br; —OH; —O—R₄; ═O; —NO₂; —NR₂R₃, wherein R₂ and R₃ are independently selected from H, and hydrocarbyl; and —NH—R₄, wherein R₄ is selected from H and hydrocarbyl; and/or
(iv) the C₂₋₂₁-alkyl-COOH is selected from the group consisting of propanoic acid, n-butanoic acid, iso-butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, optionally propanoic acid, n-butanoic acid, iso-butanoic acid, pentanoic acid, hexanoic acid, heptanoic acid, octanoic acid and nonanoic acid.

5. The composition according to claim 1, wherein the
(i) branched C₃₋₂₁-alkyl-COOH is selected from the group consisting of isobutanoic acid, isopentanoic acid, 3-methyl-pentanoic acid or 4-methyl-pentanoic acid; and/or
(ii) C₂₋₂₁-alkenyl-COOH is selected from the group consisting of 2-hexenoic acid, 3-hexenoic acid and 5-hexenoic acid.

6. The composition according to claim 1, wherein
(i) the concentration of an R₁—COOH is about 5 mM to about 100 mM, about 10 mM to about 80 mM, about 15 mM to about 70 mM, about 20 mM to about 60 mM, about 25 mM to about 50 mM, about 30 mM to about 40 mM, about 30 mM to about 35 mM; and/or
(ii) the composition is an aqueous solution; and/or
(iii) the pH of the composition is in the range of pH 6 to pH 8, pH 6 to pH 7.5, or pH 6.4 to pH 7.5, optionally wherein the composition comprises a buffer substance selected from the group consisting of ACES, ADA, BES, Bis-Tris Propane, DIPSO, EPPS, HEPPSO, Imidazol, MOBS, MES, Bis-Tris, MOPS, TRIS, MOPSO, Phosphate, PIPES, POPSO, TAPSO, TEA, TES, Tricine or HEPES buffer.

7. The composition according to claim 1 wherein the R₁—COOH or salt thereof is comprised in the composition in a concentration that it
(i) stabilizes the MHC protein comprised in the composition; and/or
(ii) increases the melting temperature of the MHC protein comprised in the composition by at least 0.5° C., 1.0° C., 1.5° C., 2.0° C., 2.5° C., 3.0° C., 3.5° C., 4.0° C., 4.5° C., 5.0° C., 5.5° C. or 6.0° C.

8. The composition according to claim 1 wherein the MHC is an MHC I or MHC II molecule, optionally a human leukocyte antigen (HLA) or a multimer of MHC I, MHC II or HLA, optionally a dimer, a trimer or a tetramer.

9. The composition according to claim 1, wherein the MHC protein is stabilized, optionally by one or more covalent bonds:
(i) between one amino acid of the alpha1 domain and one amino acid of the alpha2 domain of said stabilized MHC protein in case of MHC I; and/or
(ii) between two amino acids of the alpha1 domain of said stabilized MHC protein in case of MHC I; or
(iii) between two amino acids of the alpha1 domain or the beta1 domain of said stabilized MHC protein in case of MHC II; and/or
(iv) between one amino acid of the alpha1 domain and one amino acid of the beta1 domain of said stabilized MHC protein in case of MHC II.

10. The composition according to claim 9, wherein said covalent bond is:
(i) between α-helices, optionally between a cysteine at IMGT position 84 and a cysteine at IMGT position 139 of MHC I;
(ii) between α-helices and β-sheets of the alpha1 domain of MHC I, optionally between a cysteine at IMGT position 71 of MHC I and a cysteine at IMGT position 22 of MHC I;
(iii) between α-helices, optionally by mutating an amino acid at position 51 of MHC I and an amino acid at position 175 of MHC I into cysteines; or
(iv) between α-helices and β-sheets, optionally between a cysteine at IMGT position 71 of MHC I and a cysteine at IMGT position 22 of MHC I, and between α-helices, optionally between a cysteine at IMGT position 51 of MHC I and a cysteine at IMGT position 175 of MHC I.

11. The composition according to claim 1, wherein
(i) the MHC protein is selected from group consisting of HLA-A, HLA-B HLA-C; HLA-E; HLA-F; HLA-G; HLA-J; HLA-K, and HLA-L, wherein optionally HLA-A is selected from the group consisting of HLA-A1, HLA-A2, HLA-A3, and HLA-All, optionally HLA-A*02, optionally HLA-A*02:01; HLA-A*01:01 or HLA-A*03:01; and wherein optionally HLA-B is selected from the group consisting of HLA-B*07, HLA-B*08, HLA-B*15, HLA-B3*35 and HLA-B*44, optionally HLA-B*07:02; HLA-B*08:01, HLA-B*15:01, HLA-B*35:01 and HLA-B*44:05; and/or
(ii) one or more MHC proteins are comprised in a complex, optionally the MHC proteins are bound to beads, filaments, nanoparticles, or other carriers; and/or
(iii) the composition further comprises at least one peptide, optionally a loading peptide selected from the group consisting of glycine-methionine (GM), glycine-tyrosine (GY), glycine-leucine (GL) and glycine-phenylalanine (GF), optionally the loading peptide is GM.

12. The composition according to claim 1, wherein the MHC protein comprises or consists of SEQ ID NO: 1 and SEQ ID NO: 2.

13. A method of using an R₁—COOH or salt thereof, for stabilizing an MHC protein, wherein R₁ is substituted or unsubstituted hydrocarbyl, optionally wherein R₁ is C₂-C₂₁-hydrocarbyl.

14. A method for stabilizing an MHC protein, optionally an MHC I or MHC II, optionally an HLA, comprising the following steps:
(i) providing a solution comprising said MHC protein;
(ii) contacting said solution with an R₁—COOH or salt thereof,
wherein R₁ is substituted or unsubstituted hydrocarbyl,
optionally wherein
(1) R₁ is C₂-C₂₁-hydrocarbyl; and/or
(2) the method further comprises the step of loading the MHC with peptide, optionally an MHC-I or MHC-II peptide.

15. A kit comprising:
(i) an MHC protein; and
(ii) an R₁—COOH or salt thereof; or
(iii) the composition according to claim 1; and
(iii) optionally a peptide, optionally a loading peptide, an MHC I peptide and/or an MHC II peptide,
wherein R₁ is substituted or unsubstituted hydrocarbyl, optionally wherein R₁ is C₂-C₂₁-hydrocarbyl.


SEQUENCE LISTING (2 sequences)

SEQ ID NO: 1 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 2 - protein, 275 aa, synthetic construct
GSHSMRYFFTSVSRPGRGEPRCIAVGYVDDTQFVRFDSDAASQRMEPRAPWIEQEGPEYWDGETRKVKAHCQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE